(19) United States
(12) Patent Application Publication
(10) Pub. No.: US 2026/0232563 A1
(43) Pub. Date: Aug. 13, 2026

(54) COSMETIC OR DERMATOLOGICAL COMPOSITIONS HAVING A CUSHION EFFECT AND MODERATE-TO-LOW PILLING

(71) Applicant: ROQUETTE FRERES, Lestrem (FR)

(72) Inventors: Géraldine LOUVET-POMMIER, Lachelle (FR); Marion PERRIAU, Maule (FR)

(21) Appl. No.: 19/153,490

(22) PCT Filed: Feb. 9, 2024
(86) PCT No.: PCT/EP2024/025071

§ 371 Date: Aug. 4, 2025

(30) Foreign Application Priority Data
Feb. 9, 2023 (FR) FR2301219
Jul. 20, 2023 (FR) FR2307815

(51) Int. Cl. A61K 8/73; A61K 8/04; A61K 8/92; A61Q 19/00

(52) U.S. Cl. CPC A61K 8/732; A61K 8/042; A61K 8/922; A61Q 19/00

(57) ABSTRACT

Carboxymethylated starch is a gelling or thickening agent which can be used to obtain cosmetic or dermatological compositions having a cushion effect. However, this starch has the disadvantage of pilling during application to the skin and/or after drying. The present application provides a solution for reducing pilling by combining an oil and a selected surfactant.

### TECHNICAL FIELD

The invention relates to the field of cosmetic or dermatological formulations for topical application, comprising an aqueous phase and an oil phase, said aqueous phase being gelled by a carboxyalkylated starch.

### PRIOR ART

The cushion effect developed by the carboxyalkylated, notably carboxymethylated, starches in aqueous gels or oil-in-water emulsions is well known, as presented in the applicant's application FR3094221.

### Technical Problem

Used as thickening agents or gelling agents in aqueous gels or emulsions, notably oil-in-water, carboxymethylated starch advantageously makes it possible to obtain a cosmetic or dermatological composition giving a so-called “cushion effect” sensation when applied to the skin. Unfortunately, at the same time, the carboxymethylated starch can cause a phenomenon known as “pilling”, which consists in the formation of “pills” or “pellets”, on the skin, which prevents an even and pleasant application, and which even causes a very unpleasant sensation.

This pilling can occur both when said compositions are applied to the skin, that is when they are spread over the skin using the fingers of the hand, but also after application, when the composition has penetrated the skin and the skin is subjected to friction, either skin against skin or skin against clothing. In both cases, the pilling must be reduced to a minimum so that no residue remains on the skin.

The present application proposes a solution for reducing pilling during topical application or after drying, or during topical application and after drying, of a cosmetic or dermatological composition comprising a carboxyalkylated, notably carboxymethylated, starch, while maintaining a significant and pleasant cushion effect.

### SUMMARY OF THE INVENTION

A first object of the present application is a cosmetic or dermatological composition comprising, in a physiologically acceptable medium, an oil phase, dispersed in an aqueous phase, at least one carboxyalkylated starch or one carboxyalkylated and crosslinked starch, and at least one non-ionic, anionic or cationic surfactant, and wherein the ratio of the amount of said oil phase to the amount of said non-ionic, anionic or cationic surfactant is in the range of 0.3 to 7. Preferentially, said surfactant is a non-ionic or anionic surfactant, and more preferentially is a non-ionic surfactant.

This composition may be an oil-in-water emulsion, a water-in-oil emulsion, an aqueous or oily gel, or an aqueous or oily isotrope.

Preferentially, the composition can be an oil-in-water emulsion or an aqueous isotrope. Most preferentially, the composition is an aqueous isotrope.

The percentage by weight of carboxyalkylated starch or carboxyalkylated and crosslinked starch in said composition may be greater than or equal to 0.1%, or 0.25%, or 0.5%, or 1%, or 2%, or 3%, or 4%, or 5%, or 6%, or 7%, or 8%, by weight with respect to the total weight of said composition.

The percentage by weight of oil phase in said composition may be less than or equal to 15%, or 7%, or 6%, or 5%.

The percentage by weight of said non-ionic, anionic or cationic surfactant with respect to the total weight of said composition may be greater than or equal to 1%, or 1.5%, or 2%, or 2.5%, or 3%, or 3.5%, or 5%, or 7%.

The carboxyalkylated starch can be selected from carboxyethylated starches, carboxymethylated starches, carboxypropylated starches, carboxybutylated starches, or wherein the carboxyalkylated starch is a carboxymethylated starch. The carboxyalkylated and crosslinked starch can be selected from crosslinked carboxyethylated starches, crosslinked carboxymethylated starches, crosslinked carboxypropylated starches, crosslinked carboxybutylated starches, or the carboxyalkylated and crosslinked starch is a crosslinked carboxymethylated starch.

The anionic surfactant can be selected from salts of mixed glutamic acid and C5-C30 fatty acid anhydrides.

The non-ionic surfactant can be selected from:
alkyl glucosides,
ethoxylated sorbitan fatty acid esters,
polyethoxylated fatty alcohols,
polyglycerol esters,
mixtures consisting of at least one sorbitan ester, at least one fatty acid polyglyceryl and at least one carboxylic acid ester,
oxyethylenated and/or oxypropylated silicones.

The non-ionic surfactant can be selected from sorbitan esters, fatty acid polyglyceryls, citric acid diesters, or mixtures thereof.

The oily phase may comprise at least one oil selected from oils of natural origin, notably vegetable oils, and mineral oils, silicones, preferably the oil phase consists of at least one oil of natural origin, notably vegetable. Preferentially, the oil phase comprises at least one oil selected from:
apolar non-volatile oils,
polar non-volatile hydrocarbon oils,
non-volatile phenylated or non-phenylated silicone oils, volatile linear silicone oils,
or volatile hydrocarbon oils selected from ethers having a maximum of 16 carbon atoms.

The oil phase may comprise at least 50% by weight of a vegetable oil rich in monounsaturated fatty acids, preferentially at least 75%, preferentially at least 90%, and most preferentially consists of a vegetable oil rich in monounsaturated fatty acids.

The vegetable oil rich in monounsaturated fatty acids may comprise between 15 and 85% by weight of monounsaturated fatty acids. Preferentially, the vegetable oil rich in monounsaturated fatty acids comprises:
less than 25% by weight of saturated fatty acids,
and/or less than 70% by weight of polyunsaturated fatty acids.

The oil phase may comprise less than 50% by weight of vegetable oil, or less than 40%, or less than 30%, or less than 20%, or less than 10%, or less than 5%, or less than 1%, with respect to the total weight of said composition.

The cosmetic or dermatological composition can be a cream gel comprising a carboxyalkylated starch, a vegetable oil rich in monounsaturated fatty acids, and a non-ionic surfactant as the gelling system of said composition.

The cosmetic or dermatological composition can also comprise at least one cosmetic ingredient selected from detergent surfactants, cationic polymers, pigments, dyes, pearlescent materials and silicas.

It can take the form of a cream, a gel or a gel-cream. It can be used as a shower gel, cleansing gel, shampoo or hair conditioner.

Another object of the present application is an aqueous or hydroalcoholic gel-cream, comprising:
at least one carboxyalkylated starch, preferentially carboxymethylated,
at least one oil, preferentially a macadamia oil
and at least one non-ionic surfactant, wherein the ratio of the amount of said oil to the amount of said surfactant is in the range of 0.3 to 7.

### BRIEF DESCRIPTION OF THE DRAWINGS

Other features, details and advantages of the invention will appear from reading the following detailed description, and by analyzing the following appended figures, whereupon:

FIG. 1 shows the sensory evaluation limits for the “pilling on application” criterion.

FIG. 2 shows the sensory evaluation limits for the “pilling after drying” criterion.

### DETAILED DESCRIPTION

### Cosmetic or Dermatological Composition

The cosmetic or dermatological composition forming the subject of the present application may be a dispersion of an oil phase in an aqueous phase, said dispersion being stabilized by at least one non-ionic, anionic or cationic surfactant, and can be in the form of an oil-in-water emulsion, an oil-in-water microemulsion, an oil-in-water nanoemulsion, or an aqueous isotrope. According to one embodiment, the cosmetic or dermatological composition is an oil-in-water emulsion or an aqueous isotrope, preferentially is an aqueous isotrope. Preferentially, the surfactant is a non-ionic or anionic surfactant, more preferentially is a non-ionic surfactant.

The cosmetic or dermatological composition may also be a dispersion of an aqueous phase in an oil phase, said dispersion being stabilized by at least one non-ionic, cationic or anionic surfactant, and can be in the form of a water-in-oil emulsion, a water-in-oil microemulsion, a water-in-oil nanoemulsion, or an oily isotrope.

According to the subject of the present application, the ratio of the mass of the oil phase to the mass of the non-ionic, cationic or anionic surfactant is in a range from 0.3 to 7, or from 0.5 to 6, or from 0.75 to 5, or from 1 to 4, or from 1,25 to 3, or from 1,5 to 2.5, or is equal to 2. Preferentially, it is the ratio of the mass of the oil phase to the mass of the non-ionic or anionic surfactant which is in a range from 0.3 to 7, or from 0.5 to 6, or from 0.75 to 5, or from 1 to 4, or from 1.25 to 3, or from 1.5 to 2.5, or is equal to 2. More preferentially, it is the ratio of the mass of the oil phase to the mass of the non-ionic surfactant which is in a range from 0.3 to 7, or from 0.5 to 6, or from 0.75 to 5, or from 1 to 4, or from 1.25 to 3, or from 1.5 to 2.5, or is equal to 2.

When the cosmetic or dermatological composition is a dispersion of an oil phase in an aqueous phase, the oil phase represents from 0.5% to 65% by weight, and the aqueous phase represents from 25% to 99.5% by weight, these percentages being expressed with respect to the total weight of said composition. According to one embodiment, when the cosmetic or dermatological composition is an aqueous isotrope, the oil phase represents from 0.5% to 15% by weight, or from 0.5% to 7% by weight, or from 0.5% to 6% by weight, or from 0.5% to 5% by weight, with respect to the total weight of said composition; and the aqueous phase represents from 75% to 99.5% by weight, or from 83% to 99.5% by weight, or from 84% to 99.5% by weight, or from 85% to 99.5% by weight, with respect to the total weight of said composition.

When the cosmetic or dermatological composition is an aqueous isotrope, it may take the form of a gel or a gel-cream. When it is an oil-in-water emulsion, it can take the form of a fluid or thick cream. According to one embodiment, the cosmetic or dermatological composition forming the subject of the present application is a gel or gel-cream, preferentially a gel-cream.

The cosmetic or dermatological composition forming the subject of the present application can be used as a shower gel, cleansing gel, shampoo or hair conditioner.

According to one embodiment, the cosmetic or dermatological composition is an aqueous or hydroalcoholic gel-cream, comprising:
at least one carboxyalkylated starch or a carboxyalkylated and crosslinked starch, preferentially carboxymethylated or carboxymethylated and crosslinked,
at least one oil, preferentially a macadamia oil,
and at least one non-ionic surfactant, preferentially a mixture consisting of a sorbitan ester, a fatty acid polyglyceryl and a carboxylic acid ester, most preferentially a mixture of sorbitan laurate, polyglyceryl-4 laurate and dilauryl citrate,
   wherein the ratio of the amount of said oil to the amount of said surfactant is in the range of 0.3 to 7.

### Physiologically Acceptable Medium

“Physiologically acceptable medium” means a medium that is compatible with keratinous materials and notably skin, lips, scalp, eyelashes, eyes and/or hair.

### Aqueous Phase

The aqueous phase used in the cosmetic or dermatological composition forming the subject of the present application comprises demineralized or drinking water and at least one carboxyalkylated starch, or one carboxyalkylated and pregelatinized starch, or one carboxyalkylated and crosslinked starch.

The percentage by weight of carboxyalkylated starch, pregelatinized and carboxyalkylated starch, or carboxyalkylated and crosslinked starch, with respect to the total weight of said composition, is greater than or equal to 0.1%, or 0.25%, or 0.5%, or 1%, or 2%, or 3%, or 4%, or 5%, or 6%, or 7%. Furthermore, the percentage by weight of carboxyalkylated starch, pregelatinized and carboxyalkylated starch, or carboxyalkylated and crosslinked starch, with respect to the total weight of said composition, is less than or equal to 20%, or 18%, or 16%, or 14%, or 12%, or 10%, or 8%. Preferentially, the percentage by weight of carboxyalkylated starch, pregelatinized and carboxyalkylated starch, or carboxyalkylated and crosslinked starch is 0.1 to 20%, or 0.25 to 18%, or 0.5 to 16%, or 1 to 14%, or 2 to 12%, or 3 to 10%, or 4 to 8%, or 5 to 8%, or 6 to 8% by weight with respect to the total weight of the composition. If several pregelatinized and carboxyalkylated starches are present, the mass percentage of pregelatinized starch is the sum of the mass percentages of all the pregelatinized and carboxyalkylated starches present. If several carboxylalkylated starches are present, the mass percentage of carboxyalkylated starches is the sum of the mass percentages of all the carboxyalkylated starches present, that is the sum of carboxyalkylated starches, carboxyalkylated and crosslinked starches and carboxyalkylated and pregelatinized starches.

### Carboxyalkylated Starch

The starch used in the cosmetic or dermatological composition forming the subject of the present application is a carboxyalkylated starch, or a carboxyalkylated and crosslinked starch. This starch acts as a gelling agent for the aqueous phase. It also acts as a sensory agent with a cushion effect.

The starch may be a starch from corn, potato, wheat, rice, peas, oats, lentils, faba beans, broad beans, beans, chickpeas, cassava, or combinations thereof.

By “carboxyalkylated”, the applicant means a starch that has been chemically modified by at least one carboxyalkylation. “Carboxyalkylation”, for the purposes of the present invention, is understood notably and without this list being exhaustive, to refer to any chemical modification able to replace all or part of the 0-H hydroxyl groups of the starch with groups of the 0-(CH2)n-COOH type, of the 0-(CH2)n-COOX type, of the O—CH—COOH—(CH2)m-COOH type, or of theO—CH—COOH—(CH2)m-COOX type, wherein:
n is equal to zero (cyanidation reaction) or greater, preferably comprised between 1 (carboxymethylation reaction) and 4 (carboxybutylation reaction) and most preferentially equal to 1 (carboxymethylation) or 2 (carboxyethylation reaction or cyanoethylation reaction)
m is greater than zero and notably equal to 1 (carboxyalkylation using chloromalonic acid for example),
X is a cation, preferably monovalent, selected particularly from the group comprising sodium, potassium and lithium, in particular sodium and potassium, sodium being generally preferred.

According to one embodiment, the carboxyalkylated starch is selected from the carboxymethylated, carboxyethylated, carboxypropylated starches, or a mixture thereof. The carboxyalkylated starch used in the invention may also have been modified by at least two chemical carboxyalkylation modifications, preferentially selected from carboxymethylation, carboxyethylation, carboxypropylation.

In a particularly advantageous fashion, the carboxyalkylation consists of a carboxymethylation reaction using monochloroacetic acid or one of the salts thereof, in particular sodium monochloroacetate.

According to one embodiment, the carboxylalkylated starch is also a pregelatinized starch. It can then be selected from carboxymethylated and pregelatinized starches, carboxyethylated and pregelatinized starches, carboxypropylated and pregelatinized starches, or mixtures thereof.

Examples of carboxymethylated starch used in the invention are Beauté by Roquette® ST 118 marketed by Roquette Frères, or Vivastar® or Explotab® by JRS Pharma.

### Carboxyalkylated and Crosslinked Starch

According to one embodiment, the carboxyalkylated starch used in the subject of the present application is a carboxyalkylated and crosslinked starch. The carboxyalkylated starch may be crosslinked by a crosslinking agent selected from polyfunctional acids, polyacid anhydrides, or polyfunctional basic organic molecules, as well as the metal salts thereof.

The polyfunctional acids that have crosslinked the carboxyalkylated starch may be polycarboxylic acids, such as citric acid, or polyphosphoric acids, such as triphosphoric acid; Polyacid anhydrides that have crosslinked the carboxyalkylated starch may be mixed polyacid anhydrides such as mixed adipic/acetic anhydride. The polyfunctional basic organic molecules that have cross-linked the carboxyalkylated starch may be polyphosphates. The metal salts thereof may be salts of sodium, manganese, calcium, manganese, iron, copper, zinc. A preferred alternative cross-linking agent is sodium salts of polyacids, such as sodium trimetaphosphate, sodium tripolyphosphate. Other crosslinking agents that have crosslinked the carboxyalkylated starch may be polyfunctional aldehydes, such as glyoxal; halogenated epoxides, such as epichlorohydrin; aliphatic or aromatic diisocyanates with an alkyl chain of less than 8 carbon atoms, such as hexamethylene diisocyanate; oxohalides, such as phosphorus oxychloride.

According to one embodiment, the ratio of the amount of carboxyalkylated starch to the sum of the amount of oil phase and non-ionic, cationic or anionic surfactant, preferentially non-ionic or anionic, more preferentially non-ionic, is in the range of 0.2 to 5, preferentially from 0.4 to 3.5, preferentially from 0.5 to 2.5. If several carboxyalkylated starches are present, the amount of carboxyalkylated starch is the sum of the amounts of all carboxyalkylated starches present, that is the sum of carboxyalkylated, carboxyalkylated and crosslinked starches and carboxyalkylated and pregelatinized starches. Similarly, when several oils are present, the amount of the oil phase is the sum of the amounts of the oils present. Similarly, when several non-ionic, cationic or anionic surfactants are present, the amount of non-ionic, cationic or anionic surfactant is the sum of the amounts of non-ionic surfactants, cationic surfactants and anionic surfactants.

### Non-Ionic Surfactant

The non-ionic surfactant used in the composition forming the subject of the present application acts as a solubilizing or emulsifying surfactant of the oil phase in the aqueous phase. It has an HLB greater than or equal to 8, or 9, or 10, or 11, or 12. It can be selected from polyethoxylated, polypropoxylated or polyglycerolated alcohols, or from sorbitan esters, fatty acid polyglyceryls, citric acid diesters, alkyl polyglycosides, modified silicones, or mixtures thereof. Thus it can be selected from:
the alcohols, diols, alpha-diols and alkyl (C1-C20) phenols being polyethoxylated and/or polypropoxylated and/or polyglycerolated, and the number of ethylene oxide and/or propylene oxide groups may range from 1 to 100, and the number of glycerol groups may range from 2 to 30,
the alcohols, diols, alpha-diols and alkyl (C1-C20) phenols comprising at least one fatty chain having from 8 to 40 carbon atoms, notably from 16 to 30 carbon atoms, in particular from the alcohols comprising at least one C8 to C40 alkyl chain, saturated or unsaturated, linear or branched, oxyethylenated alcohols comprising from 1 to 100 moles of ethylene oxide, preferably from 2 to 50, more particularly from 2 to 40 moles of ethylene oxide and comprising one or two fatty chains, or mixtures of said oxyethylenated alcohols, diols, alpha-diols, alkyl (C1-C20) phenols, including: C8-C40 fatty alcohol polyethylene glycol ethers, for example pentaethylene glycol monooleyl ether, such as that marketed under the INCI name “Oleth-5”, or ricinoleic alcohol polyethylene glycol ether comprising 40 moles of ethylene oxide, such as that marketed under the INCI name “PEG-40 hydrogenated castor oil” and which is hydrogenated and oxyethylenated castor oil,
ethylene oxide and propylene oxide condensates on fatty alcohols,
polyethoxylated fatty amides preferably having from 2 to 30 ethylene oxide units, polyglycerolated fatty amides comprising on average from 1 to 5 glycerol groups and in particular from 1.5 to 4,
ethoxylated sorbitan fatty acid esters having from 2 to 100 ethylene oxide units, preferably from 2 to 40 ethylene oxide units, and at least one fatty chain comprising from 6 to 40 carbon atoms; said fatty chain being linked by an ester function to the ethoxylated sorbitan group, can be made up of a linear or branched, saturated or unsaturated carbon chain comprising from 6 to 40 carbon atoms, preferentially from 10 to 30 carbon atoms, more preferentially from 12 to 18 carbon atoms; such as polyoxyethylene sorbitan monolaurate (known under the name of Polysorbate-20), polyoxyethylene sorbitan monopalmitate (known under the name of Polysorbate-40), polyoxyethylene sorbitan monostearate (known under the name of Polysorbate-60), polyoxyethylene sorbitan monooleate (known under the name of Polysorbate-80), polyoxyethylene sorbitan trioleate (known under the name of Polysorbate-85),
sucrose fatty acid esters,
polyoxyalkylenated fatty acid esters, preferably polyoxyethylenated, having from 2 to 150 moles of ethylene oxide, including oxyethylenated vegetable oils;
N-(alkyl en C6-C24) glucamine derivatives,
amine oxides such as (C10-C14 alkyl)amine oxides or N—(C10-C14 acyl)-aminopropylmorpholine;
polyglycerol esters, also known as “fatty acid polyglyceryls”, which may be mono-, di-, or tri-esters of C6-C40 fatty acids and polyglycerol having from 2 to 100 glycerol monomers, or preferentially mono-, di-, tri-esters of C8-C30 fatty acids and polyglycerol having from 4 to 20 glycerol monomers, or more preferentially monoesters of C8-C10 fatty acids and polyglycerol having from 4 to 10 glycerol monomers, including, for example, the monoester of dodecanoic acid and decaglycerol, also known under the name of “polyglyceryl-10 laurate”, the monoester of dodecanoic acid and tetraglycerol, also known under the name of “polyglyceryl-4 laurate”, the monoester of decanoic acid and tetraglycerol, also known under the name of “polyglyceryl-4 caprate”;
oxyethylenated and/or oxypropylated silicones, which can be polydimethylsiloxanes modified with polyglycols, that is with ethylene oxide and/or with propylene oxide, such as for example PEG-12 Dimethicone,
and mixtures thereof.

According to one embodiment, the non-ionic surfactant used in the cosmetic or dermatological composition forming the subject of the present application is a mixture comprising, preferentially consisting of, a sorbitan ester, a fatty acid polyglyceryl and a citric acid diester. A non-ionic surfactant according to this embodiment is a mixture of sorbitan laurate, polyglyceryl-4 laurate and dilauryl citrate, for example that marketed under the reference “Tego® Care LTP” by Evonik Personal Care.

The non-ionic surfactant can also be selected from alkyl polyglycosides, and designated by the acronym APG. These non-ionic surfactants are well known per se. Patent FR 2 948 285 presents them in terms of structure, and explains how to prepare them. They can be represented by the following general formula (I): R1-O—(R2-O)p-(S)n,

wherein:
S represents a reducing saccharide which may comprise between 5 and 6 carbon atoms,
R1 denotes a linear or branched alkyl and/or alkenyl radical comprising approximately 6 to 24 carbon atoms, or an alkylphenyl radical in which the linear or branched alkyl group comprises approximately 8 to 24 carbon atoms,
R2 denotes an alkylene radical comprising 2 to 4 carbon atoms,
n denotes a value ranging from 1 to 15,
p denotes a value ranging from 0 to 10.

In formula (I), reducing saccharide relates to the saccharide derivatives which, in their structures, do not have a glycosidic bond established between an anomeric carbon and the oxygen of an acetal group as defined in the reference work: “Biochemistry”, Daniel Voet/Judith G. Voet, p. 250, John Wyley & Sons, 1990. The oligomeric structure(S) n, can be provided as any form of isomer, be it an optical, geometric or positional isomer; it can also represent a mixture of isomers.

In the definition of the compounds of formulas (I), S represents a reducing saccharide selected from glucose, dextrose, sucrose, fructose, idose, gulose, galactose, maltose, isomaltose, maltotriose, lactose, cellobiose, mannose, ribose, xylose, arabinose, lyxose, allose, altrose, dextrane or tallose and more particularly a reducing saccharide selected from glucose, xylose or arabinose.

A first preferred alternative of the alkyl polyglucosides according to the present invention are the C12-C20 alkyl polyglucosides, that is the compounds of formula (I) wherein:
R1 denotes more particularly a linear or branched alkyl and/or alkenyl radical comprising approximately 12 to 20 carbon atoms
p takes a value ranging from 0 to 3, preferably zero,
S denotes glucose, fructose or galactose, and more preferably glucose.

Preferably, the alkyl polyglucoside surfactant is an alkyl polyglucoside surfactant, that is an alkyl polyglucoside surfactant where S is a glucose. The glucosidic bonds between the glucose units are generally of type 1-6 or 1-4, preferably of type 1-4. The alkyl C6/C16-polyglucosides 1-4, C6/C12-polyglucosides 1-4, and notably decyl glucosides, dodecyl glucosides, hetpoglucosides also called heptyl glucosides, caprylyl glucosides, capryl glucosides and caprylyl/capryl glucosides are most particularly preferred. Another preferred alternative of the alkyl polyglucosides according to the present invention are the C12-C20 alkyl glucosides of the first preferred alternative wherein:
R1 denotes more particularly a linear alkyl radical comprising approximately 12 to 20 carbon atoms
p is equal to zero,
S denotes glucose.

Alkyl polyglucosides of formula (I) are notably commercially available under the names: Plantacare® 810 UP (R1 is C8-C10/INCI: caprylyl/capryl glucoside), Plantacare® 818 UP (R1 is C8-C16/INCI: Coco-glucoside), Plantacare® 2000 UP (R1 is C8-C16/INCI: decyl glucoside) and Plantacare® 1200 UP (R1 is C12-C16/INCI: lauryl glucoside) sold by the company BASF; Macanol® 810 (R1 is C8-C10), Macanol® 1200 (R1 is C12-C14), Macanol® 816 (mixture of R1 is C8, C10, C12, C14, C16) sold by the company FCI Technology; Neocare MF 0718 (R1 is C8-C10/INCI: caprylyl/capryl glucoside), Neocare MF 0012 (R1 is C12-C14/INCI: lauryl glucoside), Neocare MF 0002 (R1 is C8-C16/INCI: decyl glucoside), Neocare MF 818 (R1 is C8-C16/INCI: coco glucoside) sold by the company Neochem; Tego Care CG 90 (R1 is C14-C16/INCI: cetearyl glucoside) sold by the company Evonik Healthcare; the products sold by the company SEPPIC under the names ORAM IX® CG 110 (INCI: Caprylyl/Capryl Glucoside) and ORAMIX™ NS 10 (INCI: Decyl Glucoside); the products sold by the company BASF under the name Glucopon® GD 70 (INCI: C10C12-Alkyl polyglucoside).

The non-ionic surfactant of natural origin may be a mixture consisting of at least one alkyl polyglucoside and at least one fatty alcohol. In these mixtures, the alkyl polyglucosides can be selected from all the alkyl polyglucosides disclosed previously. In terms of fatty alcohols, straight or branched fatty alcohols having a total number of carbon atoms ranging from 8 to 24 are used. Mixtures of alkyl polyglucosides and fatty alcohols used in the invention and commercially available are those sold by the company SEPPIC: Montanov™ 14 (INCI: Myristyl Alcohol & Myristyl Glucoside), Montanov™ 202 (INCI: Arachidyl Alcohol and Behenyl Alcohol and Arachidyl Glucoside), Montanov™ 68 (INCI: Cetearyl Alcohol & Cetearyl Glucoside), Montanov™ 82 (INCI: Cetearyl Alcohol and Coco-Glucoside), Montanov™ S (INCI: Coco-Glucoside & Coconut Alcohol), Montanov™ L (INCI: C14-22 Alcohols & C12-20 Alkyl Glucoside).

Another variant of alkyl glucosides used in the invention is an alkyl glucoside of the formula R1-O—(R2-O)p-(S) n wherein S is glucose, R1 denotes a linear alkyl radical of 6 to 18 carbon atoms, n is 1 and p is 0. Another variant of alkyl glucosides used in the invention is a mixture of a first alkyl glucoside wherein R1 is a saturated linear alkyl radical containing 6 to 18 carbon atoms, and a second alkyl glucoside wherein R1 is a saturated linear alkyl radical containing two additional carbon atoms with respect to said first alkyl glucoside.

The non-ionic surfactant may additionally be selected from alkyl polyglycosides, preferentially alkyl polyglucosides, with an HLB according to Griffin's method, greater than or equal to 10, or greater than or equal to 12, or greater than or equal to 14.

Preferably, the non-ionic surfactant used in the cosmetic or dermatological composition forming the subject of the present application is a non-ionic surfactant that is liquid at room temperature.

When several non-ionic surfactants used in the cosmetic or dermatological composition forming the subject of the present application are present, the resulting HLB of the HLBs of all said non-ionic surfactants is in a range from 8 to 20, or from 10 to 18, or from 12 to 18, or from 14 to 18.

The mass percentage of said non-ionic surfactant with respect to the total weight of the cosmetic or dermatological composition is greater than or equal to 1%, or 1.5%, or 2%, or 2.5%, or 3%, or 3.5%, or 5%, or 7%. Furthermore, this percentage is less than or equal to 20%, or 18%, or 16%, or 14%, or 12%, or 10%, or 8%, by weight with respect to the total weight of said composition. According to one embodiment, the mass percentage in said non-ionic surfactant is from 0.5 to 10%, or from 1 to 7%, or from 1.5 to 5%, or from 2 to 3%, by weight with respect to the total weight of said composition.

### Cationic Surfactant

The cationic surfactant used in the cosmetic or dermatological composition forming the subject of the present application may be selected from C16 to C22 alkyl quaterniums, for example cetrimonium chloride, or from ester quaterniums or amidoamines.

The mass percentage of said cationic surfactant with respect to the total weight of the cosmetic or dermatological composition is greater than or equal to 1%, or 1.5%, or 2%, or 2.5%, or 3%, or 3.5%, or 5%, or 7%. Furthermore, this percentage is less than or equal to 20%, or 18%, or 16%, or 14%, or 12%, or 10%, or 8%, by weight with respect to the total weight of said composition. According to one embodiment, the mass percentage in said cationic surfactant is from 0.5 to 10%, or from 1 to 7%, or from 1.5 to 5%, or from 2 to 3%, by weight with respect to the total weight of said composition.

### Anionic Surfactant

The anionic surfactant used in the composition forming the subject of the present application acts as a solubilizing or emulsifying surfactant of the oil phase in the aqueous phase. It has an HLB greater than or equal to 8, or 9, or 10, or 11, or 12. It can be selected from salts of mixed glutamic and fatty acid anhydrides with C5-C30, preferentially C10-C25, more preferentially C15-C20, and most preferentially C18, for example sodium stearoyl glutamate.

The mass percentage of said anionic surfactant with respect to the total weight of the cosmetic or dermatological composition is greater than or equal to 1%, or 1.5%, or 2%, or 2.5%, or 3%, or 3.5%, or 5%, or 7%. Furthermore, this percentage is less than or equal to 20%, or 18%, or 16%, or 14%, or 12%, or 10%, or 8%, by weight with respect to the total weight of said composition. According to one embodiment, the mass percentage in said anionic surfactant is from 0.5 to 10%, or from 1 to 7%, or from 1.5 to 5%, or from 2 to 3%, by weight with respect to the total weight of said composition.

### Oil Phase

The oil phase used in the cosmetic or dermatological composition forming the subject of the present application comprises at least one oil and/or at least one wax. The mass percentage of said oil phase in said cosmetic or dermatological composition is less than or equal to 75%, or 50%, or 40%, or 30%, or 20%.

The oil phase may also represent a mass percentage in said cosmetic or dermatological composition of less than or equal to 15%, or 7%, or 6%, or 5%, and preferentially simultaneously greater than or equal to 0.5%, or 1%, or 2%, or 3%, by weight with respect to the total weight of said composition. Preferentially, the mass percentage of oil phase in said composition is from 0.5 to 15%, or from 1 to 7%, or from 2 to 6%, or from 3 to 5% by weight with respect to the total weight of said composition. The cosmetic or dermatological composition can then take the form of an aqueous isotrope.

The oil phase may comprise at least 50% by weight of a vegetable oil or an oil of natural origin, or at least 75%, or at least 90%, or at least 95%, or at least 99%. Preferably the oil phase consists of a vegetable oil or an oil of natural origin, preferentially a vegetable oil, and more preferentially a vegetable oil rich in monounsaturated fatty acids.

### Oil

The oil included in, or constituting, the oil phase used in the cosmetic or dermatological composition forming the subject of the present application, plays a key role in reducing pilling after drying. It can also simultaneously act as an emollient or moisturizing agent for the skin. It can be selected from volatile oils or non-volatile oils, preferentially from apolar non-volatile hydrocarbon oils, polar non-volatile hydrocarbon vegetable oils, non-volatile silicone oils, or apolar volatile hydrocarbon oils, more preferentially from polar non-volatile hydrocarbon oils, preferentially polar non-volatile hydrocarbon vegetable oils or oils of natural origin.

“Oil” is understood to mean any fatty substance in liquid form at room temperature (25° C.) and at atmospheric pressure (1,013,105 Pa).

### Non-Volatile Oils

Non-volatile refers to oils with a vapor pressure of less than 2.66 Pa, preferably less than 0.13 Pa (measured according to OECD standard 104 of 27/07/95). The non-volatile oil is selected from non-volatile silicone oils, non-volatile hydrocarbon oils, polar or apolar, and mixtures thereof; and preferably from polar non-volatile hydrocarbon oils, in particular selected from ester oils, notably described in sections (2) to (4.9) below, hydrocarbon vegetable oils, notably described in section (4.10) alone or in mixtures.

“Hydrocarbon oil” is understood to mean an oil substantially formed by, or even consisting of, carbon and hydrogen atoms, and optionally of oxygen or nitrogen atoms, and not containing a silicon or fluorine atom. Hydrocarbon oil is therefore distinct from a silicone oil and a fluorinated oil. “Silicone oil” is understood to mean an oil comprising at least one silicon atom, and notably at least one Si—O group.

### Polar Non-Volatile Hydrocarbon Oils

Polar non-volatile hydrocarbon oils are non-volatile hydrocarbon oils containing alcohol, ester, ether, carboxylic acid, amine and/or amide groups. Preferably, these oils are free of heteroatoms such as nitrogen, sulfur and phosphorus. In the present case, they comprise at least one oxygen atom. In particular, they comprise at least one alcohol functional group (it is then an “alcohol oil”) or at least one ester functional group (it is then an “ester oil”). The ester oils may be hydroxylated.

The oil used in the cosmetic or dermatological composition forming the subject of the present application is selected from the following polar non-volatile hydrocarbon oils:
(1) C10-C26 alcohols, preferably monoalcohols: C10-C26 alcohols are saturated or unsaturated, branched or unbranched, and comprise from 10 to 26 carbon atoms, preferably from 14 to 24 carbon atoms. By way of examples of fatty alcohols that can be used according to the invention, linear or branched fatty alcohols can be cited, of synthetic or even natural origin, such as, for example, alcohols originating from plant-based (copra, palm kernel, palm, etc.) or animal (tallow, etc.) materials. Of course, other long chain alcohols can also be used, such as for example, ether alcohols or even alcohols called Guerbet alcohols. Finally, some longer or shorter cuts of alcohols of natural origin also can be used, such as for example, coconut (C12 to C16) or tallow (C16 to C18) or diol or cholesterol type compounds. By way of particular examples of fatty alcohols to be preferably used, lauric, isostearylic, oleic alcohol, 2-butyloctanol, 2-undecyl pentadecanol, 2-hexyldecyl alcohol, isocetyl alcohol, octyldodecanol and mixtures thereof can be especially cited. According to an advantageous embodiment of the invention, the alcohol is selected from among octyldodecanol.
(2) the monoesters, diesters, triesters, optionally hydroxylated, of a C2-C8 mono- or polycarboxylic acid and a C2-C8 alcohol. In particular:
(2.1) monoesters of a C2-C8 carboxylic acid and of a C2-C8 alcohol, optionally hydroxylated,
(2.2) diesters of a C2-C8 carboxylic diacid and of a C2-C8 alcohol, optionally hydroxylated; such as diisopropyl adipate, diethyl-2 hexyl adipate, dibutyl adipate, 2-diethyl-hexyl succinate,
(2.3) triesters of a C2-C8 carboxylic triacid and of a C2-C8 alcohol, optionally hydroxylated, such as citric acid esters, such as trioctyl citrate, triethyl citrate, acetyltributyl citrate, tributyl citrate.
(3) esters of a C2-C8 polyol and one or more C2-C8 carboxylic acids: such as glycol and monoacid diesters, such as neopentyl glycol diheptanoate, or glycol and monoacid triesters such as triacetin.
(4) ester oils, in particular having between 17 and 70 carbon atoms: by way of example, monoesters, diesters or triesters can be cited. The ester oils may or may not be hydroxylated. The non-volatile ester oil can be selected, for example, from among:
(4.1) monoesters comprising between 17 and 40 carbon atoms in total, in particular the monoesters of formula R1-COO—R2, wherein R1 represents the residue of a linear or branched or aromatic fatty acid comprising from 4 to 40 saturated or unsaturated carbon atoms, and R2 represents a notably branched hydrocarbon chain containing from 3 to 40 carbon atoms provided that R1+R2 are greater than or equal to 17, such as, for example Purcellin (cetostearyl octanoate) oil, isononyl isononanoate, C12-C15 alcohol benzoate, 2-ethylhexyl palmitate, octyldodecyl neopentanoate, octyl-2 dodecyl stearate, octyl-2 dodecyl erucate, isostearyl isostearate, octyl-2 dodecyl benzoate, octanoates, decanoates or ricinoleates of alcohols or polyalcohols, isopropyl myristate, isopropyl palmitate, butyl stearate, hexyl laurate, 2-ethylhexyl palmitate, 2-hexyldecyl laurate, 2-octyldecyl palmitate, 2-octyldodecyl myristate. Preferably, these are esters of formula R1-COO—R2, wherein R1 represents the residue of a linear or branched fatty acid comprising from 4 to 40 carbon atoms and R2 represents a notably branched hydrocarbon chain containing from 3 to 40 carbon atoms, R1 and R2 being 10 such that R1+R2 is greater than or equal to 17. Even more specifically, the ester comprises between 17 and 40 carbon atoms in total. By way of preferred monoesters, isononyl isononanoate, isopropyl palmitate, oleyl erucate and/or octyl-2-dodecenyl neopentanoate can be cited.
(4.2) fatty acid monoesters, in particular having 18 to 22 carbon atoms, and in particular oleic acid, lauric acid, stearic acid, and diols, such as propylene glycol monostearate.
(4.3) diesters, in particular comprising between 18 and 60 carbon atoms in total, in particular between 18 and 50 carbon atoms in total. In particular, the following can be used: diesters of carboxylic diacid and monoalcohols, preferably such as diisostearyl malate; or the diesters of monocarboxylic acid and dialcohols, such as the 1,3-propanediyl ester of octanoic acid (or propanediol dicaprylate), sold under the name DUB ZENOAT by Stearinerie Dubois; or the diesters of glycol and monocarboxylic acids, such as neopentyl glycol diheptanoate, propylene glycol dioctanoate, diethylene glycol diisononanoate, or polyglyceryl-2 diisostearate (notably such as the compound sold under commercial reference DERMOL DGDIS by Alzo);
(4.4) hydroxylated monoesters and diesters, preferably having a total carbon number ranging from 18 to 70, such as polyglyceryl-3 diisostearate, isostearyl lactate, octylhydroxystearate, octyldodecyl hydroxystearate, diisostearyl malate, glycerin stearate;
(4.5) triesters, in particular comprising between 35 and 70 carbon atoms in total, in particular such as the triesters of tricarboxylic acid, such as triisostearyl citrate, or tridecyl trimellitate, or the triesters of glycol and monocarboxylic acids such as polyglycerol-2 triisostearate;
(4.6) tetraesters and pentaesters, notably having a total carbon number ranging from 35 to 70, such as penthaerythritol or polyglycerol tetraesters and a monocarboxylic acid, for example such as penthaerythrityl tetrapelargonate, tetraisostearate penthaerythrityl, penthaerythrityl tetradecanoate, glyceryl tridecyl-2 tetradecanoate, polyglyceryl-2 tetraisostearate or even penthaerythrityl tetradecyl-2 tetradecanoate; and such that dipentaerythrityl pentaesters and a monocarboxylic acid, for example dipentaerythrityl pentaisononanoate, marketed under the name of DUB VINYL by Stearinerie Dubois,
(4.7) polyesters obtained by condensation of unsaturated fatty acid dimer and/or trimer and diol such as those disclosed in patent application FR 0 853 634, such as in particular, dilinoleic acid and 1,4-butanediol. In particular, in this respect the polymer marketed by Biosynthis under the name Viscoplast 14436H (INCI name: dilinoleic acid/butanediol copolymer), or even the copolymers of polyols and diacid dimers, and their esters, such as Hailucent ISDA, can be cited;
(4.8) esters and polyesters of diol dimer and mono- or dicarboxylic acid, such as esters of diol dimer and fatty acid and esters of diol dimer and dicarboxylic acid dimer, in particular that can be obtained from a carboxylic diacid dimer particularly derived from the dimerization of an in particular C8 to C34, in particular C12 to C22, in particular C16 to C20, and more particularly C18, unsaturated fatty acid, such as esters of dilinoleic diacids and dilinoleic diol dimers, for example, such as those marketed by NIPPON FINE CHEMICAL under the trade name LUSPLAN DD-DA5® and DD-DA7®;
(4.9) polyesters resulting from the esterification of at least one triglyceride of hydroxylated carboxylic acid(s) by an aliphatic monocarboxylic acid and by an aliphatic dicarboxylic acid, optionally unsaturated such as the castor oil of succinic acid and isostearic acid marketed under the reference Zenigloss by Zénitech;
(4.10) vegetable hydrocarbon oils such as triglycerides of fatty acids (liquids at room temperature), notably fatty acids having from 7 to 40 carbon atoms, such as triglycerides of heptanoic or octanoic acids, in particular, saturated triglycerides can be cited such as caprylic/capric triglyceride and mixtures thereof, for example, such as that marketed under the reference Myritol 318 by Cognis, glyceryl triheptanoate, glycerin trioctanoate, C18-36 acid triglycerides, such as those marketed under the reference DUB TGI 24 marketed by Stearineries Dubois) the vegetable oil extracted from coconut (which is composed of caprylic/capric triglyceride), jojoba oil, macadamia oil, apricot kernel oil, as well as unsaturated triglycerides such as castor oil, olive oil, ximena oil, pracaxi oil; and other vegetable hydrocarbon oils such as Camellia Japan seed oil, avocado oil, camellia oil, hazelnut oil, tsubaki oil, cashew nut oil, argan oil, soybean oil, grape seed oil, sesame oil, corn oil, wheat germ oil, rapeseed oil, sunflower oil, cotton oil, peanut oil (rich in oleic acid (monounsaturated) and linoleic acid (di-unsaturated acid)).
(4.11) and mixtures thereof, such as, for example, oils made up of a mixture of C8-C10 fatty acid monoesters and C12-C18 fatty alcohols, such as MIGLYOL Coco 810 by IOI Oleo GmbH (INCI name: coco-Caprylate/Caprate).

Preferably, the polar non-volatile hydrocarbon oil is selected from ester oils, and in particular monoesters comprising at least 17 carbon atoms in total, pentaesters, notably having at least 35 carbon atoms, and from vegetable hydrocarbon oils, as well as mixtures thereof.

### Apolar Non-Volatile Hydrocarbon Oils

With respect to apolar non-volatile oils, also called “mineral oils” as they originate from the distillation of fossil fuels such as oil or coal, it is possible to cite more specifically paraffin oil, squalane, pentadecane, nonadecane, eicosane, isoeicosane, polybutenes, which may or may not be hydrogenated, hydrogenated or non-hydrogenated polyisoprenes, hydrogenated or non-hydrogenated polydecenes, decene/butene copolymers, polybutene/polyisobutene copolymers, and mixtures thereof. These oils are composed of hydrocarbons, mainly alkanes or alkenes with 15 to 40 carbon atoms. An example of a mixture of apolar non-volatile hydrocarbon oils is Emogreen L15 marketed by Seppic, which is a mixture of C15-C19 alkanes. Preferentially, the cosmetic or dermatological composition comprises an apolar non-volatile hydrocarbon oil which is paraffin.

### Non-Volatile Silicone Oils

With respect to silicone non-volatile oils, non-volatile, non-phenylated silicone oils can be cited, for example such as polydimethylsiloxane, also called dimethicone, or phenylated, such as for example polyphenylmethylsiloxane. Preferentially, the non-volatile silicone oil is selected from non-phenylated linear silicone oils, such as polydimethylsiloxane.

Phenylated silicone oils, such as, for example, diphenyl dimethicone, phenyl trimethicone, trimethylsiloxyl phenyl dimethicone, diphenylsiloxy phenyl trimethicone, trimethyl pentaphenyl trisiloxane, or tetramethyl tetraphenyl trisiloxane, and mixtures thereof also can be cited. Advantageously, the non-volatile silicone oil does not comprise (an) oxyalkylenated C2-C3 group(s) (oxyethylenated, oxypropylenated) or (a) glycerolated group(s).

### Volatile Oils

“Volatile oil” refers to oils with a non-zero vapor pressure at room temperature and atmospheric pressure, notably having a vapor pressure ranging from 2.66 Pa to 40,000 Pa, in particular from 2.66 Pa to 13,000 Pa, and more particularly from 2.66 Pa to 1300 Pa.

The volatile oils can be hydrocarbon or silicone.

In particular, apolar volatile hydrocarbon oils having from 8 to 16 carbon atoms can be cited like C8-C16 branched alkanes such as C8-C16 iso-alkanes (also called isoparaffins), isododecane, isodecane, isohexadecane and, for example, oils sold under the trade names of Isopars or Permetyls. Preferably, the volatile hydrocarbon oil is selected from among volatile hydrocarbon oils having from 8 to 16 carbon atoms and mixtures thereof, in particular from isododecane, isodecane, isohexadecane, and in particular is isohexadecane. It is also possible to cite volatile linear alkanes comprising from 8 to 16 carbon atoms, in particular from 10 to 15 carbon atoms, and more particularly from 11 to 13 carbon atoms, for example, such as n-dodecane (C12) and n-tetradecane (C14) sold by Sasol under references PARAFOL 12-97 and PARAFOL 14-97, respectively, as well as mixtures thereof, the undecane-tridecane mixture, such as Cetiol Ultimate by BASF, the mixtures of n-undecane (CH) and n-tridecane (C13) obtained in Examples 1 and 2 of the application WO 2008/155059 by the company Cognis, and mixtures thereof, as well as ethers having at most 16 carbon atoms, such as for example dioctyl ether known as “dicaprylyl ether”, with polypropylene glycol butyl ether having 14 proplyene units, also referred to as “PPG-14 butyl ether”.

As silicone volatile oils, linear silicone volatile oils can be cited such as hexamethyldisiloxane, octamethyltrisiloxane, decamethyltetrasiloxane, tetradecamethylhexasiloxane, hexadecamethylheptasiloxane and dodecamethyl-pentasiloxane. Hexamethylcyclotrisiloxane, octamethylcylotetrasiloxane, decamethylcyclopenta-siloxane, dodecamethylcyclohexasiloxane, cyclopentasiloxane and cyclohexasiloxane can be cited as cyclic silicone volatile oils.

### Waxes:

The oil phase used in the cosmetic or dermatological composition forming the subject of the present application may comprise at least one silicone wax, or a polar or apolar hydrocarbon wax. The wax is generally a solid lipophilic compound at room temperature (25° C.), with a reversible solid/liquid state change, having a melting point that is in particular greater than or equal to 30° C., more particularly greater than 45° C. Advantageously, the melting point is less than or equal to 90° C., more particularly less than or equal to 80° C., and preferably less than or equal to 70° C. The melting point of a solid fatty substance can be measured using a differential scanning calorimeter (DSC), for example the calorimeter sold under the name “DSC Q100” by the company TA Instruments with the “TA Universal Analysis” software.

The measurement protocol is the following: a sample of solid fatty substance of approximately 5 mg is placed in an “aluminum hermetic capsule” crucible. The sample undergoes a first temperature increase ranging from 20° C. to 120° C., at a heating rate of 2° C./minute up to 80° C., then is left in the isotherm at 100° C. for 20 minutes, then is cooled from 120° C. to 0° C. at a cooling rate of 2° C./minute, and finally undergoes a second temperature increase ranging from 0° C. to 20° C. at a heating rate of 2° C./minute. The melting temperature value of the solid fatty substance is the value of the top of the most endothermic peak of the observed melting curve, representing the variation in the difference in absorbed power based on the temperature.

### Polar Hydrocarbon Waxes

More specifically, the polar wax is selected from among ester hydrocarbon waxes, alcohol hydrocarbon waxes, silicone waxes, and mixtures thereof. “Hydrocarbon wax” is understood to be a wax substantially formed by, or even consisting of, carbon and hydrogen atoms, and optionally of oxygen or nitrogen atoms, and not containing a silicon atom or fluorine. It can contain alcohol, ester, ether, carboxylic acid, amine and/or amide groups. “Ester wax” is understood to mean a wax comprising at least one ester functional group. The ester waxes can also be hydroxylated. “Alcohol wax” is understood to mean a wax comprising at least one alcohol functional group, in other words comprising at least one free hydroxyl group (OH). The additional alcohol wax in particular does not comprise an ester functional group. “Silicone wax” is understood to mean a wax comprising at least one silicon atom, and in particular comprising Si—O groups.

### Ester Waxes:

In particular, the following can be used as ester wax:
i) the waxes of formula R1-COO—R2, wherein R1 and R2 represent linear, branched or cyclic aliphatic chains, of which the number of atoms varies from 10 to 50, which can contain a heteroatom, in particular oxygen, and of which the melting point temperature varies from 30° C. to 120° C., preferably from 30° C. to 100° C. In particular, as ester wax, a C20-C40 alkyl (hydroxystearyloxy) stearate (the alkyl group comprising from 20 to 40 carbon atoms), alone or in a mixture, or a C20-C40 alkyl stearate can be used. Such waxes are particularly sold under the names “Kester Wax K 82 P®”, “Hydroxypolyester K 82 PR”, “Kester Wax K 80 PR”, or “KESTER WAX K82H” by KOSTER KEUNEN. Mixtures of esters of C14-C18 carboxylic acids and alcohols also can be used, such as the “Cetyl Ester Wax 814” products by KOSTER KEUNEN, “SP Crodamol MS MBAL”, “Crodamol MS PA” by CRODA, “Miraceti” by LASERSON. A glycol and butylene glycol montanate (octacosanoate) also can be used such as the LICOWAX KPS FLAKES (INCI name: glycol montanate) wax sold by Clariant.
ii) the di-(trimethylol-1, 1, 1 propane) tetrastearate, sold under the name Hest 2T4S® by HETERENE.
iii) the diester waxes of a carboxylic diacid of general formula R3-(—OCO—R4-COO—R5), wherein R3 and R5 are identical or different, preferably identical and represent a C4-C30 alkyl group (alkyl group comprising from 4 to 30 carbon atoms 35) and R4 represents a linear or branched C4-C30 aliphatic group (alkyl group comprising from 4 to 30 carbon atoms) and which may or may not contain one or more unsaturated bonds.
   Preferably, the C4-C30 aliphatic group is linear and unsaturated.
iv) the waxes obtained by catalytic hydrogenation of animal or plant-based oils, in particular having C8-C32 linear or branched fatty chains, for example, such as hydrogenated jojoba oil, hydrogenated sunflower oil, hydrogenated castor oil, hydrogenated coprah oil, and waxes obtained by hydrogenation of castor oil esterified with cetyl alcohol, such as those sold under the names of Phytowax ricin 16L64® and 22L73® by SOPHIM. Such waxes are described in application FR-A-2792190. The waxes sold under the name “PHYTOWAX Olive 18 L 57” can be cited as waxes obtained by hydrogenation of olive oil esterified with stearyl alcohol.
v) the waxes of animal or plant origin, such as beeswax, synthetic beeswax, carnauba wax, candelilla wax, lanolin wax, rice bran wax, Ouricury wax, Alfa wax, berry wax, shellac wax, cork fiber wax, sugar cane wax, Japan wax, sumac wax, montan wax, orange and lemon wax, Laurier wax, hydrogenated jojoba wax, sunflower wax, in particular refined.
vi) hydrocarbon, polyoxyalkylenated or polyglycerolated, natural or synthetic, waxes of animal or plant origin also can be cited; the number of oxyalkylenated units (C2-C4) can vary from 2 to 100, the number of glycerolated units can vary from 1 to 20. By way of example, polyoxyethylenated beeswaxes can be cited, such as PEG-6 beeswax, PEG-8 beeswax; polyoxyethylenated carnauba waxes, such as PEG-12 carnauba; hydrogenated or non-hydrogenated, polyoxyethenated or polyoxypropylenated, lanolin waxes, such as PEG-30 lanolin, PEG-75 lanolin; PPG-5 lanolin wax glyceride; polyglycerolated beeswaxes, in particular polyglyceryl-3 Beeswax, the mixture of Acacia Decurrens/Jojoba/Sunflower Seed Wax/Polyglyceryl-3 Esters, polyglycerolated plant-based waxes such as mimosa, jojoba, sunflower waxes, and mixtures thereof (Acacia Decurrens/Jojoba/Sunflower Seed Wax Polyglyceryl-3 Esters).
vii) the waxes corresponding to the partial or total esters, preferably total, of a C16-C30 carboxylic acid, that is saturated, optionally hydroxylated, with glycerol. Total esters means that all the hydroxylated functional groups of the glycerol are esterified. By way of example, trihydroxystearin (or glyceryl trihydroxystearate), tristearin (or glyceryl tristearate), tribehenin (or glyceryl tribehenate), alone or as a mixture, can be cited. Among suitable compounds, glycerol triesters and 12-hydroxystearic acid, or hydrogenated castor oil, can be cited, such as, for example, Thixcin R, Thixcin E, marketed by Elementis Specialties.
viii) and mixtures thereof.

### Alcohol Waxes

As an alcohol wax, alcohols, preferably linear, preferably saturated, can be cited comprising from 16 to 60 carbon atoms, the melting point of which ranges between 25° C. and 90° C. By way of examples of alcohol wax, stearic alcohol, cetyl alcohol, myristic alcohol, palmitic alcohol, behenic alcohol, erucic alcohol, arachidyl alcohol, or mixtures thereof, can be cited.

### Apolar Hydrocarbon Waxes

The wax can be selected from apolar hydrocarbon waxes. “Apolar hydrocarbon wax” is understood to mean a wax comprising only carbon or hydrogen atoms in its structure. In other words, such a wax is free of other atoms, in particular heteroatoms such as, for example, nitrogen, oxygen, silicon. By way of an illustration of apolar waxes suitable for the invention, hydrocarbon waxes such as microcrystalline waxes, paraffin waxes, ozokerite, polymethylene waxes, polyethylene waxes, waxes obtained by Fischer-Tropsch synthesis, microwaxes, in particular polyethylene, can be cited in particular.

### Silicone Waxes

Examples of silicon waxes include: mixtures comprising a compound of the C30-45 Alkyldimethylsilyl Polypropylsilsesquioxane type (INCI name), such as the product Dow Corning SW-8005 C30 Resin Wax marketed by the company Dow Corning; mixtures comprising a compound of the C30-45 Alkyl Methicone type (INCI name), such as the product Dow Corning® AMS-C30 Cosmetic Wax; mixtures comprising a C20-24 alkyl methicone compound, such as KCC Beauty's SeraSoft SW 73; mixtures comprising a C26-28 alkyl methicone compound, such as Wacker's Belsil® CDM 3526 VP and Belsil® CM 7026 VP. Silicone beeswax also can be cited.

The cosmetic or dermatological cosmetic composition forming the subject of the present application may comprise a content of preferably polar, preferably polar hydrocarbon wax(es) of between 0.5 and 10% by weight, or from 0.5 to 6% by weight, or from 1 to 4% by weight, with respect to the weight of said composition.

### Vegetable Oil Rich in Monounsaturated Fatty Acids

“Vegetable oil rich in monounsaturated fatty acids” is understood to mean a vegetable oil, or an oil of vegetable origin, which comprises between 15 and 95% by weight of monounsaturated fatty acids, the expression “in monounsaturated fatty acids” meaning “in a monounsaturated fatty acid” or “in at least one monounsaturated fatty acid”, preferentially between 30 and 90%, more preferentially between 50 and 85%, with respect to the total weight of said oil. This oil may also comprise less than 25% by weight of saturated fatty acid, the expression “of saturated fatty acid” meaning “of a saturated fatty acid” or “of at least one saturated fatty acid”, and/or less than 70% by weight of polyunsaturated fatty acid, with respect to total weight of said oil, the expression “of polyunsaturated fatty acid” meaning “of a polyunsaturated fatty acid” or “of at least one polyunsaturated fatty acid”.

The monounsaturated fatty acid may be oleic acid, palmitoleic acid, gadoleic acid (or 11-eicosenoic acid), or mixtures thereof. The polyunsaturated fatty acid may be linoleic acid, alpha-linoleic acid or mixtures thereof. The saturated fatty acid may be palmitic acid, stearic acid, arachidic acid or mixtures thereof.

Vegetable oils rich in monounsaturated fatty acids used in the composition forming the subject of the present application are macadamia oil, olive oil, sweet almond oil and sunflower seed oil. According to one embodiment, the vegetable oil rich in monounsaturated fatty acid is macadamia oil.

### Embodiments of the Composition According to the Invention

According to one embodiment, the oil is selected from:
apolar non-volatile oils,
polar non-volatile hydrocarbon oils,
   non-volatile phenylated or non-phenylated silicone oils, volatile linear silicone oils,
   or volatile hydrocarbon oils selected from ethers having a maximum of 16 carbon atoms.

According to a preferential embodiment, the oil is selected from apolar non-volatile oils, vegetable hydrocarbon oils, or non-phenylated non-volatile silicone oils.

According to a more preferential embodiment, the oil is selected from apolar non-volatile oils or vegetable hydrocarbon oils.

According to a more preferential embodiment, the oil is selected from apolar non-volatile oils or vegetable oils rich in monounsaturated fatty acids.

According to one embodiment, the oil is selected from:
apolar non-volatile hydrocarbon oils,
polar non-volatile hydrocarbon vegetable oils, with the exception of olive oil,
polar non-volatile linear non-vegetable hydrocarbon oils,
non-volatile linear silicone oils, preferentially non-phenylated,
apolar volatile hydrocarbon oils of the ether type having a single ether function.

This embodiment achieves a cushion effect ranging from 6.5 to 8.5 together with pilling on application less than or equal to 5 and pilling after drying less than or equal to 5.

According to one embodiment, the oil is selected from:
apolar non-volatile hydrocarbon oils,
polar non-volatile hydrocarbon vegetable oils, with the exception of olive oil and sunflower oil,
non-volatile linear silicone oils, preferentially non-phenylated.

This embodiment achieves a cushion effect ranging from 7 to 8.5 together with pilling on application less than or equal to 5 and pilling after drying less than or equal to 4.

According to one embodiment, the oil is selected from:
apolar non-volatile hydrocarbon oils,
polar non-volatile hydrocarbon vegetable oils, with the exception of olive oil, sunflower oil, argan oil and caprylyl/capryl triglyceride.

This embodiment achieves a cushion effect ranging from 7 to 8.5 together with pilling on application less than or equal to 4.5 and pilling after drying less than or equal to 2.75.

According to one embodiment, the oil is selected from:

apolar non-volatile oils,
fatty acid triglycerides, macadamia oil, sweet almond oil, jojoba oil, argan oil, sunflower oil, olive oil,
non-volatile phenylated or non-phenylated linear silicone oils, or non-volatile linear silicone oils,
or volatile hydrocarbon oils selected from ethers having a maximum of 16 carbon atoms.

According to one embodiment, the oil is selected from:
apolar non-volatile oils,
saturated triglycerides, macadamia oil, sweet almond oil, jojoba oil, argan oil, sunflower oil,
non-volatile phenylated or non-phenylated linear silicone oils, preferentially non-volatile, non-phenylated linear silicone oils,
or volatile hydrocarbon oils selected from ethers having a maximum of 16 carbon atoms.

According to one embodiment, the oil is selected from:
apolar non-volatile oils,
saturated triglycerides, macadamia oil, sweet almond oil, jojoba oil, argan oil,
or non-volatile non-phenylated linear silicone oils or non-volatile linear silicone oils.

According to one embodiment, the oil is selected from:
apolar non-volatile oils,
macadamia oil, sweet almond oil, jojoba oil, argan oil,
non-volatile, non-phenylated linear silicone oils or volatile linear silicone oils.

According to one embodiment, the oil is selected from:
apolar non-volatile oils,
macadamia oil, sweet almond oil, jojoba oil, argan oil,
non-volatile, non-phenylated linear silicone oils.

According to one embodiment, the oil used in the cosmetic or dermatological composition forming the subject of the present application is selected from:
apolar non-volatile oils,
macadamia oil, sweet almond oil or jojoba oil.

According to one embodiment, the oil is selected from:
paraffin oil,
caprylic/capric triglyceride, macadamia oil, sweet almond oil, jojoba oil, argan oil, sunflower oil,
dimethicone,
or dioctyl ether.

This embodiment achieves a cushion effect ranging from 6.5 to 8.5 together with pilling on application less than or equal to 5 and pilling after drying less than or equal to 5.

According to one embodiment, the oil is selected from:
paraffin oil,
macadamia oil, sweet almond oil, jojoba oil, argan oil,
or dimethicone.

This embodiment achieves a cushion effect ranging from 7 to 8.5 together with pilling on application less than or equal to 5 and pilling after drying less than or equal to 3.5.

According to one embodiment, the oil is selected from:
paraffin oil,
macadamia oil, sweet almond oil or jojoba oil.

This embodiment achieves a cushion effect ranging from 7 to 8.5 together with pilling on application less than or equal to 4.5 and pilling after drying less than or equal to 2.75.

According to one embodiment, the surfactant is non-ionic and is selected from:
alkyl glucosides,
ethoxylated sorbitan fatty acid esters,
polyethoxylated fatty alcohols,
polyglycerol esters,
mixtures consisting of at least one sorbitan ester, at least one fatty acid polyglyceryl and at least one carboxylic acid ester, preferentially mixtures consisting of a sorbitan ester, a fatty acid polyglyceryl and a carboxylic acid ester, and most preferentially a mixture of sorbitan laurate, polyglyceryl-4 laurate and dilauryl citrate,
oxyethylenated and/or oxypropylated silicones.

According to one embodiment, the surfactant is selected from:
salts of mixed glutamic acid and C5-C30 fatty acid anhydrides,
alkyl glucosides,
polyethoxylated fatty alcohols,
mixtures consisting of at least one sorbitan ester, at least one fatty acid polyglyceryl and at least one carboxylic acid ester, preferentially mixtures consisting of a sorbitan ester, a fatty acid polyglyceryl and a carboxylic acid ester, and most preferentially a mixture of sorbitan laurate, polyglyceryl-4 laurate and dilauryl citrate.

According to one embodiment, the surfactant is anionic and is selected from salts of mixed glutamic acid and C5-C30 fatty acid anhydrides.

According to one embodiment, the surfactant is selected from:
sodium salts of C5-C30, preferentially C10-C25, more preferentially C15-C20, and most preferentially C18, mixed glutamic acid and fatty acid anhydrides,
alkyl glucosides of formula R1-O—(R2-O) p-(S) n wherein S is glucose, R1 denotes a linear alkyl radical of 6 to 18 carbon atoms, n is equal to 1, and p is equal to 0; or a mixture of an alkyl glucoside wherein R1 is a saturated linear alkyl radical containing 6 to 18 carbon atoms, and a second alkyl glucoside wherein R1 is a saturated linear alkyl radical containing two additional carbon atoms with respect to said first alkyl glucoside,
ethoxylated sorbitan fatty acid esters having 2 to 100 ethylene oxide units and one to three fatty chains comprising 10 to 30 carbon atoms,
polyethylene glycol and C8-C40 fatty alcohol ethers,
mono-, di-, or tri-esters of C6-C40 fatty acids and polyglycerol having from 2 to 100 glycerol monomers, selected from: mono-, di- or tri-esters of up to C8 fatty acids and polyglycerol having at most 3 glycerol monomers; mono-, di- or tri-esters of up to C10 fatty acids and polyglycerol having 4 glycerol monomers; or mono-, di- or tri-esters of up to C12 fatty acids and polyglycerol having at least 5 glycerol monomers,
mixtures consisting of at least one sorbitan ester, at least one fatty acid polyglyceryl and at least one citric acid diester, preferentially mixtures consisting of a sorbitan ester, a fatty acid polyglyceryl and a carboxylic acid ester, and most preferentially a mixture of sorbitan laurate, polyglyceryl-4 laurate and dilauryl citrate,
polydimethylsiloxanes modified with ethylene oxide and/or propylene oxide.

According to one embodiment, the surfactant is selected from:
sodium salts of C5-C30, preferentially C10-C25, more preferentially C15-C20, and most preferentially C18, mixed glutamic acid and fatty acid anhydrides,
alkyl glucosides of formula R1-O—(R2-O) p-(S) n wherein S is glucose, R1 denotes a linear alkyl radical of 6 to 18 carbon atoms, n is equal to 1, and p is equal to 0; or a mixture of an alkyl glucoside wherein R1 is a saturated linear alkyl radical containing 6 to 18 carbon atoms, and a second alkyl glucoside wherein R1 is a saturated linear alkyl radical containing two additional carbon atoms with respect to said first alkyl glucoside,
ethoxylated sorbitan fatty acid esters having preferably at least 5 ethylene oxide units and from one to three fatty chains comprising from 12 to 18 carbon atoms, more preferentially having between 5 and 40 ethylene oxide units and from one to three fatty chains comprising from 12 to 18 carbon atoms, even more preferentially having 20 ethylene oxide units and from one to three fatty chains comprising from 12 to 18 carbon atoms, and most preferentially having 20 ethylene oxide units and one fatty chain comprising from 12 to 18 carbon atoms,
polyethylene glycol and C8-C40 fatty alcohol ethers,
mono-, di-, or tri-esters of C6-C40 fatty acids and polyglycerol having from 2 to 100 glycerol monomers, selected from: mono-, di- or tri-esters of up to C8 fatty acids and polyglycerol having at most 3 glycerol monomers; mono-, di- or tri-esters of up to C10 fatty acids and polyglycerol having 4 glycerol monomers; or mono-, di- or tri-esters of up to C12 fatty acids and polyglycerol having at least 5 glycerol monomers,
mixtures consisting of at least one sorbitan ester, at least one fatty acid polyglyceryl and at least one citric acid diester, preferentially mixtures consisting of a sorbitan ester, a fatty acid polyglyceryl and a carboxylic acid ester, and most preferentially a mixture of sorbitan laurate, polyglyceryl-4 laurate and dilauryl citrate,
ethylene oxide-modified polydimethylsiloxanes having at least 12 ethylene oxide units.

According to one embodiment, the surfactant is selected from:
sodium salts of C5-C30, preferentially C10-C25, more preferentially C15-C20, and most preferentially C18, mixed glutamic acid and fatty acid anhydrides,
alkyl glucosides of formula R1-O—(R2-O) p-(S) n wherein S is glucose, R1 denotes a linear alkyl radical of 8 to 10 carbon atoms, n is equal to 1, and p is equal to 0; or a mixture of an alkyl glucoside wherein R1 is a saturated linear alkyl radical containing 8 carbon atoms, and an alkyl glucoside wherein R1 is a saturated linear alkyl radical containing 10 carbon atoms,
ethoxylated sorbitan fatty acid esters preferably having 20 ethylene oxide units and a fatty chain comprising 12 to 18 carbon atoms,
mixtures consisting of a sorbitan ester, a fatty acid polyglyceryl and a carboxylic acid ester, and most preferentially a mixture of sorbitan laurate, polyglyceryl-4 laurate and dilauryl citrate.

This embodiment achieves a cushion effect ranging from 6.25 to 8.25 together with pilling on application less than or equal to 3.75 and pilling after drying less than or equal to 1.75.

According to a preferential embodiment, the surfactant is selected from sodium stearoyl glutamate, caprylyl/capryl glucoside, heptyl glucoside, polyoxyethylene sorbitan monolaurate (polysorbate-20), polyoxyethylene sorbitan monooleate (polysorbate-80), polyoxyethylene sorbitan trioleate (polysorbate-85), PEG-80 sorbitan laurate, polyethylene glycol ricinoleic alcohol ether comprising 40 moles of ethylene oxide (PEG-40 hydrogenated castor oil), decaglycerol and dodecanoic acid monoester (polyglyceryl-10 laurate), decanoic acid and tetraglycerol monoester (polyglyceryl-4 caprate), pentaethylene glycol monooleyl ether (oleth-5), Tego Care LTP MB, oxyethylenated and oxypropylenated polydimethylsiloxane (PEG-12 dimethicone). This embodiment achieves a cushion effect ranging from 6.25 to 8.25 together with pilling on application less than or equal to 4.75 and pilling after drying less than or equal to 4.0.

According to a preferential embodiment, the surfactant is selected from sodium stearoyl glutamate, caprylyl/capryl glucoside, heptyl glucoside, polyoxyethylene sorbitan monolaurate, polyoxyethylene sorbitan monooleate, polyoxyethylene sorbitan trioleate, polyethylene glycol ricinoleic alcohol ether comprising 40 moles of ethylene oxide, dodecanoic acid decaglycerol monoester, decanoic acid tetraglycerol monoester, pentaethylene glycol monooleyl ether, mixtures of sorbitan laurate, polyglyceryl-4 laurate and dilauryl citrate. This embodiment achieves a cushion effect ranging from 6.25 to 8.25 together with pilling on application less than or equal to 4.75 and pilling after drying less than or equal to 3.0.

According to a very preferential embodiment, the surfactant is selected from sodium stearoyl glutamate, caprylyl/capryl glucoside, polyoxyethylene sorbitan monolaurate or polyoxyethylene sorbitan monooleate. This embodiment achieves a cushion effect ranging from 6.25 to 8.25 together with pilling on application less than or equal to 3.75 and pilling after drying less than or equal to 1.75.

### Effects on the Sensory Profile

The cosmetic or dermatological composition forming the subject of the present application has the advantage of a sensory profile characterized by a significant cushion effect and by moderate pilling on application and/or after drying. “Significant cushion effect” is understood to mean a cushion effect, measured according to the sensory evaluation method described in the present application, having a rating greater than or equal to 5, preferentially greater than or equal to 6, greater than or equal to 7, and most preferentially greater than or equal to 7.5. “Moderate pilling on application” and “moderate pilling after drying” means pilling measured according to the sensory evaluation method described in the present application, having a rating less than or equal to 5, preferentially less than or equal to 4, preferentially less than or equal to 3.

When the cosmetic or dermatological composition is applied, two types of residue may form: wet pills and/or a film of wet material, which may remain on the skin after application. “Film of wet material” refers to the layer of cosmetic or dermatological composition that has not penetrated the skin, and is therefore more or less evenly spread over the skin, with a thickness of less than about 1 mm, preferentially less than or equal to about 0.5 mm. “Wet pills” refers to fragments of cosmetic or dermatological composition having sufficient consistency to be individually visible and felt to the touch, which may resemble aggregates or agglomerates of the constituents of said composition, and which still have a high water content.

After penetration or drying of the residues formed during application, rubbing the skin with the index finger can result in the formation of two types of residue: a “sand-like” residue and/or dry pills. “Sand-like residue” refers to more or less dry fragments, less than 1 mm in size, which may resemble small scales or fine to very fine sand, and which are felt as a slight scrubbing sensation when rubbed into the skin. “Dry pills” refers to low-water aggregates in the form of individualized and well-formed “pellets” containing all or some of the constituents of the cosmetic or dermatological composition. These dry pills can take the form of “strands” greater than or equal to 2 mm in length and greater than or equal to 0.5 mm in width.

According to one embodiment, the cosmetic or dermatological composition has:
a cushion effect having a rating greater than or equal to 7, preferentially greater than or equal to 7.5 and,
pilling on application having a rating less than or equal to 5, preferentially less than or equal to 4.5, and most preferentially less than or equal to 4 and,
pilling after drying having a rating less than or equal to 4, preferentially less than or equal to 3, and most preferentially less than or equal to 2.5.

### Use of Oil and Surfactant to Reduce Pilling

Another object of the present application is the use of an oil and a surfactant in a cosmetic or dermatological composition comprising at least one carboxyalkylated starch as a thickening agent for the aqueous phase of said composition, said use being characterized in that the ratio of the mass of the oil to the mass of the surfactant is in a range from 0.3 to 7, or from 0.5 to 6, or from 0.75 to 5, or from 1 to 4, or from 1.25 to 3, or from 1.5 to 2.5, or is equal to 2, and said use making it possible to reduce or eliminate pilling on application and/or after drying, while maintaining a cushion effect.

According to one embodiment of this use, it consists in the use of a polar non-volatile hydrocarbon vegetable oil or oil of vegetable origin and a non-ionic surfactant. The polar non-volatile hydrocarbon vegetable oil can be selected from vegetable oils rich in monounsaturated fatty acids, macadamia oil, for example, macadamia oil. The non-ionic surfactant can be selected from sorbitan esters, polyglyceryl esters of fatty acids, citric acid diesters, or mixtures thereof, for example the mixture of sorbitan laurate, polyglyceryl-4 laurate and dilauryl citrate, for example that marketed under the reference “Tego® Care LTP” by Evonik Personal Care.

Gelling System with Cushion Effect and Moderate Pilling

Another object of the present application is a mixture of a carboxyalkylated starch, a surfactant and an oil as a gelling or thickening system with a cushion effect and moderate pilling, used for preparing cosmetic or dermatological compositions having a cushion effect and moderate pilling on application and/or after drying.

According to one embodiment, the gelling or thickening system with a cushion effect and moderate pilling comprises, preferentially consists of:
at least one carboxyalkylated starch, preferentially selected from carboxyethylated starches, carboxymethylated starches, carboxypropylated starches, carboxybutylated starches, and most preferentially the carboxyalkylated starch is a carboxymethylated starch,
at least one oil, preferentially selected from oils of natural origin, notably vegetable, and mineral oils, silicones, preferably the oil phase consists of at least one oil of natural origin, notably vegetable,
at least one surfactant, preferentially selected from non-ionic surfactants or cationic surfactants.

In said gelling or thickening system, the ratio of the mass of oil to the mass of surfactant ranges from 0.3 to 7, or from 0.5 to 6, or from 0.75 to 5, or from 1 to 4, or from 1.25 to 3, or from 1.5 to 2.5, or is equal to 2.

### Sensory Profile Evaluation Method

The sensory profile of a cosmetic or dermatological composition according to the subject of the present application is evaluated by a panel of at least four people with expertise in the sensory analysis of cosmetic products, trained for four months, at a rate of one session per week, to evaluate the following three sensory criteria according to a scale ranging from 0 to 10: cushion effect, pilling on application and pilling after drying. The “0” limit on the scale for a sensory criterion corresponds to the minimum value of said criterion, that is the weakest sensation for this criterion, and the “10” limit corresponds to the maximum value of said criterion, that is the strongest sensation for this criterion.

Panelists are trained to memorize the sensations they feel when they hold and apply to their forearms reference compositions corresponding to the extreme limits of the ranges of the criteria to be evaluated, namely:
for the “0” limit of the three criteria evaluated herein: the “fluid” limit of the “EBITouch®” kit for the “cushion effect” criterion, and bare skin on the inner forearm area, with little or no hair, for pilling on application and pilling after drying.
for the “10” limit of the three criteria evaluated herein: an aqueous gel of Beauté by Roquette® ST 118 carboxymethylated starch at 8% by weight with respect to the total weight of the gel, and Beauté by Roquette PO 071 sorbitol at 10% by weight with respect to the total weight of the gel, prepared in demineralized water according to the following protocol: in demineralized water maintained at 80° C., dissolve sorbitol under stirring at 100-200 rpm with a deflocculating stirrer blade for 5 minutes, then disperse carboxymethylated starch under stirring at 500-1000 rpm with a deflocculating stirrer blade and maintain under stirring for 15 minutes at 80° C.

The three criteria are then evaluated according to the following protocols:

For the “cushion effect” criterion: this is evaluated by holding approximately 0.04 grams of the reference composition or the composition to be evaluated in the hand, placing the composition between the index finger and thumb, then applying three squeezes with the fingers no more than 0.5 cm apart. Limit “10” is described as giving a “bounce” effect, and corresponding to a highly elastic composition that retains its consistency. Limit “0” is described as giving no bounce effect, and corresponding to a composition which is not at all elastic, and which is fluid, that is which spreads without retaining its consistency.

For the “pilling on application” criterion: this is evaluated when approximately 0.2 grams of composition are applied to the inside of the forearm. The composition is picked up with the index finger and then applied to a rectangular so-called “measurement” area on the inside of the forearm, measuring about 7 cm lengthways and about 5 cm crossways, by performing ten rotations with the tip of the index finger, covering the entire surface and at a speed of around one revolution per second. Limit “10” is described as forming a large amount of wet pilling. Rating “4” corresponds to the maximum amount of “film of wet material” residue remaining on the skin surface after application. Limit “0” corresponds to the total absence of wet pilling and of the “film of wet material” type residues.

For the “pilling after drying” criterion: this is evaluated seven minutes after the evaluation of the “pilling on application” criterion on the same rectangular area “as is”, that is in the state it is in when the “pilling on application” criterion is evaluated, therefore after the application of about 0.2 grams of composition to the forearm according to the protocol of the “pilling on application” criterion. The waiting time of seven minutes must allow the penetration and/or the drying of the composition which may have persisted in the form of a film of wet material and/or wet pilling at the end of the application step. After the seven-minute waiting time, the index finger is rubbed over the rectangular measurement area, moving back and forth five times in the longitudinal direction of the forearm, and applying strong pressure to the area in question. Limit “0” corresponds to the total absence of “sand-like” residue and dry pilling. Rating “4” corresponds to a maximum amount of “sand-like” residue. Rating “6” corresponds to the appearance of the first dry pilling, which is generally concomitant with the disappearance of the “sand-like” residue, a priori due to its transformation into dry pilling. Limit “10” is described as forming a large amount of dry pilling.

To support the training sessions followed and the evaluations carried out by the panelists, criteria evaluation flowcharts were drawn up so that the panelists could give consistent ratings. The following tables show the ratings proposed in these flowcharts and the associated descriptions. The right-hand column shows the level of acceptability of the ratings for an average consumer.

TABLE 1
Cushion effect

| Rating | Description | |
|---|---|---|
| 0 | Not elastic, | Not |
| | no material remains between the fingers, the composition spreads | acceptable |
| | to the sides or becomes fluid. | |
| 1 | Not elastic, | |
| | very little material remains between the fingers, the composition | |
| | spreads to the sides or flows freely. | |
| 2 | Not elastic, | |
| | a little material remains between the fingers, | |
| | some of the composition spreads to the sides or flows freely, some | |
| | spreads less to the sides and is less fluid. | |
| 3 | Not elastic, | |
| | a moderate amount of material remains between the fingers, the | |
| | composition does not spread to the sides and does not flow freely | |
| 4 | Not elastic, | |
| | a lot of material remains between the fingers, the composition does | |
| | not spread to the sides and does not flow freely | |
| 5 | Slightly elastic, | |
| | very little material remains between the fingers, the composition | |
| | spreads to the sides or flows freely. | |
| 6 | Slightly elastic, | Acceptable |
| | little material remains between the fingers, | |
| | some of the composition spreads to the sides or flows freely, some | |
| | spreads less to the sides and is less fluid. | |
| 7 | Slightly elastic, | |
| | a moderate amount of material remains between the fingers, the | |
| | composition does not spread to the sides and does not flow freely | |
| | OR very elastic, | |
| | no or very little material remains between the fingers, the | |
| | composition spreads to the sides or flows freely. | |
| 8 | Not very elastic, a lot of material remains between the fingers, the | |
| | composition does not spread to the sides or flow freely OR Very | |
| | elastic, a little material remains between the fingers, some of the | |
| | composition spreads to the sides or flows freely, some spreads out | |
| | less to the sides and is less fluid. | |
| 9 | Very elastic, | |
| | a moderate amount of material remains between the fingers, the | |
| | composition does not spread to the sides and does not flow freely | |
| 10 | Very elastic, a lot of material remains between the fingers, the | |
| | composition does not spread to the sides and does not flow freely | |

TABLE 2
Pilling on application

| Rating | Description | |
|---|---|---|
| 0 | No layer of material is observed, the product penetrates upon | Acceptable |
| | application | |
| 1 | A thin layer of homogeneous material remains on the skin | |
| 2 | A moderate layer of homogeneous material is observed; this layer | |
| | may be translucent | |
| 3 | A thick homogeneous layer of material remains on the skin, but | |
| | does not pill; this layer may be slightly white | |
| 4 | A very thick homogeneous layer of material remains on the skin, | |
| | but does not pill; this layer may be slightly white. | |
| 5 | A rating of “5” is not permitted. The panelist must choose between | |
| | a rating of 4 or 6. | |
| 6 | Pilling occurs and does not disappear or penetrate during rotations; | Not |
| | the number of pills is less than or equal to 2, | acceptable |
| | these pills have a “length” less than or equal to 2 mm, and a “width” | |
| | less than or equal to 0.5 mm | |
| 7 | Pilling occurs and does not disappear or penetrate during rotations; | |
| | the number of pills is less than or equal to 4, | |
| | these pills have a “length” less than or equal to 2 mm, and a “width” | |
| | less than or equal to 0.5 mm | |
| 8 | Pilling occurs and does not disappear or penetrate during rotations; | |
| | the number of pills is less than or equal to 6, these pills have a | |
| | “length” between 2 mm and 4 mm, and a “width” less than or equal | |
| | to 0.5 mm | |
| 9 | Pilling occurs and does not disappear or penetrate during rotations; | |
| | the number of pills is less than or equal to 8, | |
| | these pills have a “length” between 2 and 4 mm, and a “width” less | |
| | than or equal to 0.5 mm | |
| 10 | Pilling occurs and does not disappear or penetrate during rotations; | |
| | the number of pills is greater than or equal to 10, | |
| | these pills have a “length” greater than 4 mm, and a “width” greater | |
| | than 0.5 mm | |

TABLE 3
Pilling after drying

| Rating | Description | |
|---|---|---|
| 0 | no pilling or sand-like residue is observed | Acceptable |
| 1 | no thick pilling is observed; a few fine residues resembling “sand” | |
| | are felt to the touch, and are not visible to the naked eye on the | |
| | skin of the forearm | |
| 2 | no thick pilling is observed; a few fine residues resembling “sand” | |
| | are felt to the touch, but are barely visible to the naked eye on the | |
| | skin of the forearm, and are visible to the naked eye on the index | |
| | finger after rubbing | |
| 3 | no thick pilling is observed; a few fine residues resembling “sand” | |
| | are felt to the touch, and are visible to the naked eye on the skin of | |
| | the forearm and on the index finger after rubbing | |
| 4 | no thick pilling is observed; a few fine residues resembling “sand” | |
| | are felt to the touch, and are visible to the naked eye on the skin of | |
| | the forearm and on the index finger after rubbing; the residues | |
| | cover a larger surface area than rating 3 | |
| 5 | A rating of “5” is not permitted. The panelist must choose between | |
| | a rating of 4 or 6. | |
| 6 | Pilling occurs; a low number of pills form and are small in size: | Not |
| | there are less than 3 pills, and the length of the pills is less than or | acceptable |
| | equal to 1 mm, and the width is less than or equal to 0.5 mm | |
| | Pilling occurs; a moderate number of pills form and are small in | |
| | size: there are between 4 and 10 pills, and the length of the pills is | |
| | less than or equal to 1 mm, and the width is less than or equal to | |
| | 0.5 mm | |
| 8 | Pilling occurs; a moderate number of pills form and are medium in | |
| | size: there are between 4 and 10 pills, and the length of the pills is | |
| | greater than or equal to 3 mm, and the width is greater than or | |
| | equal to 1 mm | |
| 9 | Pilling occurs; a moderate number of pills forms and they are large | |
| | in size: there are between 10 and 14 pills, and the length of the | |
| | pills is greater than or equal to 3 mm, and the width is greater than | |
| | or equal to 1 mm | |
| 10 | Thick pilling occurs; a large number of pills form and they are large | |
| | in size: there are more than 15 pills, and the length of the pills is | |
| | greater than or equal to 3 mm, and the width is greater than or | |
| | equal to 1 mm | |

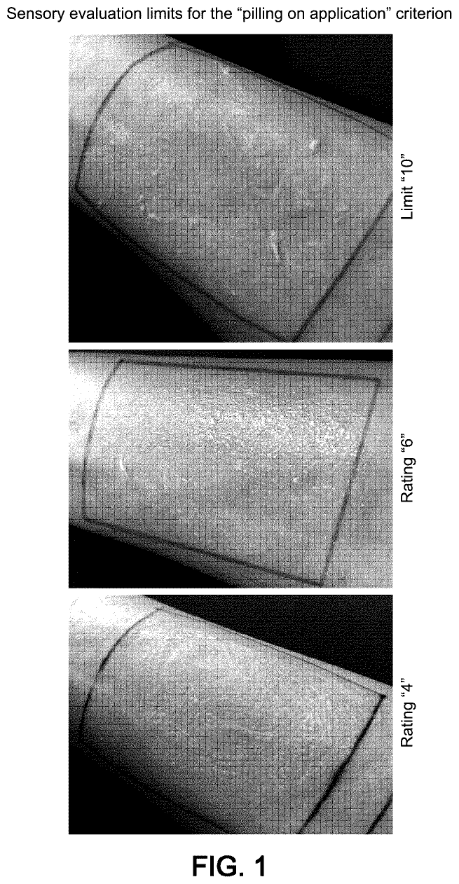

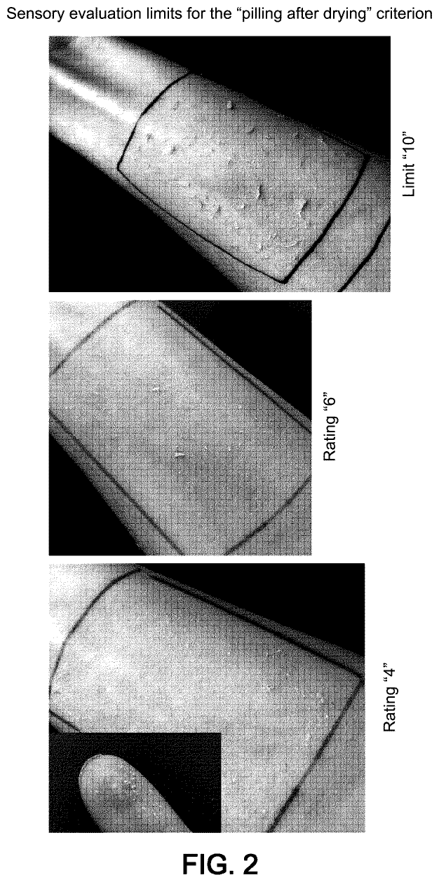

These tables are also shown as a flow chart in FIGS. 1, 2 and 3.

### Examples

### Example 1: Reducing Pilling of a Carboxymethylated Aqueous Starch Gel by Adding Macadamia Oil and Tego® Care LTP

In this example, we studied the impact of introducing macadamia oil and a non-ionic liquid surfactant at room temperature, Tego® Care LTP from producer @Evonik Industries AG, on the sensory profile of an aqueous gel of the carboxymethylated starch Beauté by Roquette® ST 118 (abbreviated as “BBR ST 118”) at 7% starch by weight with respect to the total weight of the gel. By means of experimental screening along two dimensions, namely the macadamia oil content and the Tego® Care LTP content, we determined the sensory profiles of BBR ST 118 aqueous gels on the sensory criteria of the cushion effect, pilling on application and pilling after drying, according to the method set out in the present application.

To prepare aqueous gels of BBR ST 118 with different contents of macadamia oil and Tego® Care LTP, we proceeded according to the following protocol, according to the contents shown in the tables of results below:
in demineralized water previously heated to 25° C., disperse Beauté by Roquette@ ST 118 carboxymethylated starch under stirring at 2500 rpm with a deflocculating stirrer blade, and maintain under stirring and temperature for 15 minutes,
under stirring and at 25° C., simultaneously add the previously mixed oil and surfactant,
maintain under stirring at 2500 rpm with the deflocculating stirrer blade for 10 minutes at 25° C.,
add a preservative and homogenize for one minute under stirring.

Sensory evaluations of each gel were then carried out within a week of preparation of said gels to avoid any variations during the storage of said gels. The results obtained are presented in the following tables (empty boxes correspond to untested combinations).

TABLE 4

| | | Cushion effect | | | | | | | | | |
|---|---|---|---|---|---|---|---|---|---|---|---|
| Macadamia | 7 | | | | | | | | 7 | | |
| oil (% by | 6 | | | | | | | | | | |
| weight) | 5 | 8.4 | | 8 | 7 | 7.5 | 7 | | | | |
| | 3 | | | | 7.1 | | 8 | | | | |
| | 2 | 7.1 | | | | | | | | | |
| | 1 | | 8.8 | | | | 8.3 | | | | |
| | 0 | 8.9 | | 8.9 | | | | 8.9 | | 8.9 | 6.7 |
| | | 0 | 0.5 | 1 | 1.5 | 2 | 2.5 | 3 | 3.5 | 5 | 7 |

Tego Care LTP (% by weight)

According to the results presented in Table 4, macadamia oil alone or Tego Care LTP alone have a slight negative impact on the cushion effect, for a particular macadamia oil content value of 2%, and for a Tego Care LTP content of 7%. When combined, macadamia oil and Tego Care LTP cause a reduction in the cushion effect from at least 2% oil and at least 1.5% Tego Care LTP: the cushion effect rating drops below 8. However, this is still an acceptable rating, which gives a perceptible cushion effect.

TABLE 5

| | | Pilling on application | | | | | | | | | |
|---|---|---|---|---|---|---|---|---|---|---|---|
| Macadamia | 7 | | | | | | | | 3 | | |
| oil (% by | 6 | | | | | | | | | | |
| weight) | 5 | 5.1 | | 3.2 | 4 | 4.7 | 3.8 | | | | |
| | 3 | | | | 4.3 | | 3.8 | | | | |
| | 2 | 2.4 | | | | | | | | | |
| | 1 | | 1.6 | | | | 3.1 | | | | |
| | 0 | 1.6 | | 3.8 | | | | 3.3 | | 3.8 | 4.3 |
| | | 0 | 0.5 | 1 | 1.5 | 2 | 2.5 | 3 | 3.5 | 5 | 7 |

Tego Care LTP (% by weight)

According to the results presented in Table 5, regarding pilling on application, BBR ST 118 aqueous gel has an already low rating, with a value of 1.6. The introduction of macadamia oil or Tego Care LTP alone leads to an increase in pilling on application: ratings on this criterion reach values close to the acceptable limit of 5, for contents of 5% oil or 7% Tego Care LTP. Similarly, the combination of macadamia oil and Tego Care LTP also leads to an increase in pilling on application, but to a lesser extent, so that a rating of about 3 or 4 is achievable.

TABLE 6

| | | Pilling after drying | | | | | | | | | |
|---|---|---|---|---|---|---|---|---|---|---|---|
| Macadamia | 7 | | | | | | | | 1.4 | | |
| oil (% by | 6 | | | | | | | | | | |
| weight) | 5 | 8 | | 4 | 2.3 | 2.3 | 2 | | | | |
| | 3 | | | | 3.6 | | 3 | | | | |
| | 2 | 8 | | | | | | | | | |
| | 1 | | 8.8 | | | | 5.1 | | | | |
| | 0 | 8.1 | | 7.9 | | | | 6.3 | | 5.4 | 5.3 |
| | | 0 | 0.5 | 1 | 1.5 | 2 | 2.5 | 3 | 3.5 | 5 | 7 |

Tego Care LTP (% by weight)

According to the results presented in Table 6, regarding pilling after drying, macadamia oil alone does not reduce pilling after drying, which is evaluated at 8 up to 5% oil. Tego Care LTP helps reduce pilling after drying, significantly from a Tego Care LTP content of at least 3% by weight, to achieve ratings of less than about 6. Surprisingly and unexpectedly, the combination of macadamia oil and Tego Care LTP very significantly reduces pilling after drying. By combining at least 3% oil by weight with respect to the total weight of the composition, and at least 1.5% Tego Care LTP by weight with respect to the total weight of the composition, pilling after drying is reduced to ratings of less than 4. Ratings of less than 2 are even achieved for combinations of at least 5% oil and at least 2.5% Tego Care LTP.

For these combinations of oil and Tego Care LTP content, pilling on application remains less than or equal to ratings of 4 or even 3, according to the results in Table 5. Furthermore, the cushion effect remains greater than or equal to 7 for these same combinations, which is an entirely acceptable rating, indicating a significantly perceptible cushion effect.

This example therefore shows that the combination of macadamia oil and Tego Care LTP non-ionic surfactant reduces pilling after drying, while maintaining pilling on application and the cushion effect at acceptable ratings.

### Example 2: Combinations of Tego Care LTP with Other Oils

In this example, aqueous gels comprising 6 to 8% by weight of BBR ST 118 carboxymethylated starch were evaluated, to which a binary combination of Tego Care LTP non-ionic surfactant and an oil selected from the oils in Table 7-1 was added. Initially, the surfactant content was set at 2.5% by weight, and the oil content at 5% by weight, with respect to the total weight of an aqueous gel at 7% by weight of BBR ST 118.

TABLE 7-1

| Oil type | Trade name - Supplier | INCI name |
|---|---|---|
| Vegetable no1 | Macadamia oil - Cooper | Macadamia Ternifolia |
| | | seed oil |
| Vegetable no2 | Refined sweet almond oil - Cooper | Prunus Amygdalus |
| | | Dulcis Oil |
| Vegetable no3 | Jojoba oil - Cooper | Simmondsia Chinensis |
| | | (Jojoba) Seed Oil |
| Vegetable no4 | Argan oil - Cooper | Argania Spinosa |
| | | (Argan) Kernel Oil |
| Vegetable no5 | Virgin sunflower oil - Cooper | Helianthus Annuus |
| | | Seed Oil |
| Vegetable no6 | Olive oil - Aromazone | Olea Europaea Fruit |
| | | Oil |
| Ether no1 | Cetiol ® OE - BASF | Dicaprylyl Ether |
| Ether no2 | TEGOSOFT ® PBE BF - Evonik | PPG-14 Butyl Ether |
| Ester no1 | DUB ™ VINYL - | Dipentaerythrityl |
| | Stearinerie Dubois | Pentaisononan oate |
| Ester no2 | DUB ™ MCT 55/45 MB - Stearinerie | Caprylic/capric |
| | Dubois | triglycerides |
| Ester no3 | TEGOSOFT ® INI - Evonik | Isononyl isononanoate |
| Ester no4 | Isopropyl Myristate - BASF | Isopropyl Myristate |
| Alkane no1 | Cetiol@ Ultimate - BASF | Undecane, Tridecane |
| Alkane no2 | Isohexadecane - IMCD | Isohexadecane |
| Silicon no1 | XIAMETER ™ PMX-200 Silicone | Dimethicone |
| | Fluid - Dow Chemical Company | |
| Silicone no2 | XIAMETER ™ PMX- 0345 | Cyclopentasiloxane, |
| (cyclic) | Cyclosiloxane Blend - Dow Chemical | Cyclohexas iloxane |
| | Company | |
| Mineral no1 | Carnation ® - Sonneborn | Paraffinum liquidum |

Each gel is then subjected to a sensory evaluation by the trained panel on the criteria of cushion effect, pilling on application and pilling after drying. The results of these sensory evaluation which were performed are presented in Table 7-2.

TABLE 7-2

| | Cushion | Pilling on | Pilling after | Viscosity | |
|---|---|---|---|---|---|
| Oil reference | effect | application | drying | (mPa · s) | Color |
| Mineral no1 | 8.22 | 3.56 | 2.56 | 43,375 | Opaque |
| | | | | | white ++ |
| Vegetable no1 | 7.9 | 4.2 | 2.2 | 29,425 | Opaque white ++ |
| Vegetable no2 | 7 | 4.25 | 2.5 | 29,075 | Opaque |
| | | | | | white + |
| Vegetable no3 | 7.5 | 4 | 2.25 | 26,750 | Opaque |
| | | | | | yellow |
| Vegetable no4 | 7.1 | 4.7 | 3.1 | 30,300 | Opaque |
| | | | | | white + |
| Silicon no1 | 8 | 4.45 | 3.36 | 33,425 | Slightly |
| | | | | | opaque |
| Ester no2 | 6.7 | 4.4 | 4 | 31,200 | Opaque |
| | | | | | white + |
| Ether no1 | 6.91 | 4.82 | 5 | 24,275 | Opaque white +++ |
| Vegetable no5 | 18.25 | 5 | 3.75 | 32,500 | Opaque white ++ |
| Vegetable no6 | 7.25 | 5.25 | 2.25 | 26,775 | Opaque |
| | | | | | yellow |
| Etherno2 | 8.36 | 5.27 | 3.09 | 28,775 | Opaque |
| | | | | | white |
| Ester no1 | 7.1 | 5.3 | 4.2 | 33 000 | Opaque white ++ |
| Ester no3 | 7.67 | 6.08 | 4.5 | 48,900 | Opaque |
| | | | | | white +++ |
| Silicone no2 | 7.82 | 6.09 | 3.45 | 43,525 | Opaque |
| (cyclic) | | | | | white |
| Ester no4 | 8.5 | 5.5 | 4.92 | 43,525 | Opaque |
| | | | | | white ++ |
| Alkane no1 | 6.83 | 3.75 | 3.75 | 21,450 | Opaque |
| | | | | unstable | white +++ |
| Alkane no2 | 6.83 | 4.5 | 5.42 | 23,825 | Opaque |
| | | | | unstable | white +++ |
| Reference | 8.9 | 4.7 | 8 | 40,275 | Translucent |
| BBR ST 118 | | | | | |

Oils in the first row (Mineral oil n°1) to the ninth row (Vegetable oil n°5) all achieve a cushion effect greater than or equal to 6.7 and pilling on application less than 5.0. Together, these same oils achieve pilling after drying less than or equal to 5.0. Oils in the tenth row (Vegetable oil n°6) to the seventeenth row (Alkane oil n°2) give pilling on application greater than 5, or even 6, and/or pilling after drying greater than 5, and/or form unstable creams.

### Example 3: Combinations of Macadamia Oil with Other Surfactants

In this example, aqueous gels comprising 6 to 8% by weight of BBR ST 118 carboxymethylated starch are evaluated, to which a binary combination of macadamia oil and a surfactant selected from the surfactants in Table 8-1 is added. Initially, the surfactant content is set at 2.5% by weight and the oil content at 5% by weight, with respect to the total weight of the 7% by weight BBR ST 118 aqueous gel.

TABLE 8-1

| Surfactant | | |
|---|---|---|
| reference | Trade name - Supplier | INCI name |
| T1 | TEGO ® Care LTP - | Sorbitan Laurate, Polyglyceryl-4 |
| | Evonik | Laurate, Dilauryl Citrate |
| T2 | TEGO ® SML 20 - Evonik | Polysorbate 20 |
| T3 | Tween ™ 80 - Croda | Polysorbate 80 |
| T4 | MONTANOX ™ 85 VG | Polysorbate 85 |
| | DF - Seppic | |
| T5 | Microcare ® Silicone | PEG-12 Dimethicone |
| | E1200 - Thor | |
| T6 | Eumulgin ® CO 40 - | PEG-40 Hydrogenated Castor Oil |
| | BASF | |
| T7 | Sisterna SP70-C - | Sucrose stearate |
| | Sisterna | |
| T8 | MONTANE ™ 20 - Seppic | Sorbitan Laurate |
| T9 | Tween ™ 28 - Croda | PEG-80 Sorbitan Laurate |
| T10 | TEGO ® Care CG 90 - | Cetearyl Glucoside |
| | Evonik | |
| T11 | SEPICLEAR ™ G7 - | Heptyl Glucoside |
| | Seppic | |
| T12 | ORAMIX ™ CG 110 - | Caprylyl/Capryl Glucoside |
| | Seppic | |
| T13 | IMWITOR ® 948 - 101 | Glyceryl Oleate |
| | Oleo | |
| T14 | Dermofeel ® Gsc Sg - | Glyceryl Stearate Citrate |
| | Evonik | |
| T15 | Hydriol PGCH.4- Hydrior | Polyglyceryl-4 Caprate |
| | AG | |
| T16 | Durosoft ® PG4L-SG - | Polyglyceryl-4 Laurate |
| | Stephenson | |
| T17 | IMWITOR ® 600- IOI | Polyglyceryl-3 Polyricinoleate |
| | Oleo | |
| T18 | NIKKOL Decaglyn ® | Polyglyceryl-10 Laurate |
| | 1 - L - NIKKOL | |
| T19 | Eumulgin ® SG - BASF | Sodium Stearoyl Glutamate |
| T20 | EUMULGIN ® O 5 - | Oleth-5 |
| | BASF | |
| T21 | DEHYQUART ® 40 - | Cetrimonium Chloride |
| | BASF | |

Each gel is then subjected to a sensory evaluation by the trained panel on the criteria of cushion effect, pilling on application, and pilling after drying. The results of these sensory evaluations which were performed are presented in Table 8-2.

TABLE 8-2

| Surfactant | Cushion | Pilling on | Pilling after | Viscosity | |
|---|---|---|---|---|---|
| reference | effect | application | drying | (mPa · s) | Color |
| T19 | 6.33 | 2.83 | 10.67 | 15,333 | Opaque |
| | | | | | white ++ |
| T12 | 8.17 | 3.67 | 0.67 | 24,400 | Opaque |
| | | | | | white |
| T2 | 6.60 | 1.40 | 0.80 | 22,233 | Opaque |
| | | | | | white ++ |
| T3 | 6.80 | 1.40 | 1.60 | 20,900 | Opaque |
| | | | | | white ++ |
| T6 | 6.78 | 1.44 | 2.11 | 21,167 | Opaque |
| | | | | | white ++ |
| T18 | 7.33 | 3.00 | 2.17 | 23,733 | Opaque |
| | | | | | white +++ |
| T15 | 7.64 | 3.27 | 2.45 | 25,867 | Opaque |
| | | | | | white + |
| T20 | 7.50 | 1.33 | 2.50 | 23,800 | Opaque |
| | | | | | white +++ |
| T1 | 7.78 | 4.56 | 12.56 | 27,633 | Opaque |
| | | | | | white + |
| T11 | 7.10 | 2.60 | 2.60 | 29,033 | Opaque |
| | | | | | white |
| T4 | 7.20 | 4.20 | 2.80 | 23,200 | Opaque |
| | | | | | white ++ |
| T9 | 7.00 | 2.73 | 3.55 | 24,100 | Opaque |
| | | | | | white ++ |
| T5 | 8.20 | 1.60 | 3.80 | 21,800 | Opaque |
| | | | | | white +++ |
| T16 | 7.43 | 5.57 | 2.29 | 32,833 | Opaque |
| | | | | | white |
| T8 | 7.55 | 5.82 | 3.27 | 26,267 | Opaque |
| | | | | | white |
| T7 | 8.00 | 7.00 | 3.82 | 40,267 | Opaque |
| | | | | | white ++ |
| T21 | 3.83 | 8.00 | 1.50 | 32,167 | Opaque |
| | | | | | white +++ |
| T17 | 9.29 | 3.57 | 6.00 | 153,367 | Opaque |
| | | | | | white +++ |
| T13 | 4.00 | 5.83 | 6.67 | 122,033 | Opaque |
| | | | | | white ++ |
| Reference | 8.90 | 4.70 | 8.00 | 40,275 | Translucent |
| BBR ST 118 | | | | | |

Surfactants in the first row (surfactant T19) to the thirteenth row (surfactant T5) all achieve a cushion effect greater than or equal to 6.3 and pilling on application less than 5.0. Together, these same surfactants achieve pilling after drying less than or equal to 4.0. Surfactants in the fourteenth row (surfactant T16) to the nineteenth row (surfactant T13) give pilling on application greater than 5, or even 7, and/or pilling on drying greater than 6.

1. A cosmetic or dermatological composition comprising in a physiologically acceptable medium, an oil phase dispersed in an aqueous phase, at least one carboxyalkylated starch or a carboxyalkylated and crosslinked starch, and at least one non-ionic, anionic or cationic surfactant, preferentially at least one non-ionic or anionic surfactant, and wherein the ratio of the amount of said oil phase to the amount of said non-ionic, anionic or cationic surfactant, preferentially non-ionic or anionic, is in the range of 0.3 to 7.

2. The composition according to claim 1, wherein the composition is an oil-in-water emulsion, a water-in-oil emulsion, an aqueous or oily gel, or an aqueous or oily isotrope, preferentially an oil-in-water emulsion or an aqueous isotrope.

3. The composition according to claim 1, wherein the mass percentage of carboxyalkylated, or carboxyalkylated and crosslinked starch, in said composition is greater than or equal to 0.1%, or 0.25%, or 0.5%, or 1%, or 2%, or 3%, or 4%, or 5%, or 6%, or 7%, or 8%, by weight with respect to the total weight of said composition.

4. The composition according to claim 3, wherein the mass percentage of carboxyalkylated, or carboxyalkylated and crosslinked starch is less than or equal to 20%, or 18%, or 16%, or 14%, or 12%, or 10%, or 8%.

5. The composition according to claim 1, the mass percentage of oil phase in said composition is less than or equal to 15%, or 7%, or 6%, or 5%.

6. The composition according to claim 5, wherein the mass percentage of oil phase in said composition is greater than or equal to 0.5%, or 1%, or 2%, or 3%, by weight with respect to the total weight of said composition.

7. The composition according to claim 1, wherein the mass percentage of said non-ionic, anionic or cationic surfactant with respect to the total weight of said composition is greater than or equal to 1%, or 1.5%, or 2%, or 2.5%, or 3%, or 3.5%, or 5%, or 7%, preferentially is from 0.5 to 10%, or from 1 to 7%, or from 1.5 to 5%, or from 2 to 3%.

8. The composition according to claim 1, wherein the ratio of the amount of said carboxyalkylated starch or said carboxyalkylated and crosslinked starch to the sum of the amount of said oil phase and of said surfactant is in the range from 0.2 to 5, preferentially from 0.4 to 3.5, preferentially from 0.5 to 2.5.

9. The composition according to claim 1, wherein the carboxyalkylated starch, or the carboxyalkylated and crosslinked starch, is selected from carboxyethylated starches, carboxymethylated starches, carboxypropylated starches, carboxybutylated starches, or wherein the carboxyalkylated starch is a carboxymethylated starch.

10. (canceled)

11. The composition according to claim 1, wherein the surfactant is non-ionic and is selected from:
alkyl glucosides,
ethoxylated sorbitan fatty acid esters,
polyethoxylated fatty alcohols,
polyglycerol esters,
mixtures consisting of at least one sorbitan ester, at least one fatty acid polyglyceryl and at least one carboxylic acid ester,
oxyethylenated and/or oxypropylated silicones.

12. The composition according to claim 1, wherein the surfactant is anionic and is selected from salts of mixed glutamic acid and C5-C30 fatty acid anhydrides.

13. (canceled)

14. The composition according to claim 1, wherein
the oil phase comprises at least one oil selected from:
apolar non-volatile oils,
polar non-volatile hydrocarbon oils,
non-volatile phenylated or non-phenylated silicone oils, volatile linear silicone oils,
or volatile hydrocarbon oils selected from ethers having a maximum of 16 carbon atoms.

15. The composition according to claim 1, wherein the oil phase comprises at least 50% by weight of a vegetable oil rich in monounsaturated fatty acids, preferentially at least 75%, preferentially at least 90%, and most preferentially consists of a vegetable oil rich in monounsaturated fatty acids.

16. The composition according to claim 1, wherein the vegetable oil rich in monounsaturated fatty acids comprises between 15 and 85% by weight of monounsaturated fatty acids.

17. The composition according to claim 1, wherein the vegetable oil rich in monounsaturated fatty acids comprises:
less than 25% by weight of saturated fatty acids,
and/or less than 70% by weight of polyunsaturated fatty acids.

18-23. (canceled)
